(19) United States
(12) Patent Application Publication
(10) Pub. No.: US 2004/0005649 A1
(43) Pub. Date: Jan. 8, 2004

(54) Anti-stilbene antibodies

(76) Inventors: Peter Wirsching, Del Mar, CA (US); Kim D Janda, La Jolla, CA (US); Richard A. Lerner, La Jolla, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(21) Appl. No.: 10/363,182

(22) PCT Filed: Sep. 13, 2001
(86) PCT No.: PCT/US01/42160

(51) Int. Cl. G01N 33/53; C07K 16/00; G01N 33/537; G01N 33/543; A61K 49/00; C07H 15/04

(52) U.S. Cl. 435/7.92; 530/387.1; 424/9.1; 536/120; 536/4.1

(57) ABSTRACT

The present invention provides an anti-stilbene antibody. Preferably, the antibody is a monoclonal antibody. Exemplary and preferred such antibodies are designated herein as 19G2, 20F2, 21C6, 22B9, 25F8, 25E2, 23E4, 23G3, 23D3, 23C2, 25C10, 24B6, 21E2, 16H10 and 9E11. The present invention further provides hybridomas that produce and secrete anti-stilbene antibodies. An antibody of the present invention has particular utility in processes for identifying and/or locating target moieties appended to or incorporating antigenic stilbene. In one embodiment, therefore, the present invention further provides a method of detecting antigenic stilbene. The method includes the steps of exposing antigenic stilbene to an anti-stilbene antibody and detecting an anti-stilbene antibody-stilbene immunoconjugate. Such immunoconjugates can be detected using fluoroscopic procedures.

Funds used to support some of the studies reported herein were provided by the National Institutes of Health (GM4385, AI39089 and P01CA27489). The United States Government, therefore, has certain rights in this invention.

### TECHNICAL FIELD OF THE INVENTION

The field of this invention is antibodies. More particularly, the present invention pertains to anti-stilbene antibodies that have use in the localization of targeted moieties.

### BACKGROUND OF THE INVENTION

Over the past 15 years, catalytic antibody technology has ushered in a renaissance of understanding protein interactions with small organic molecules. The principle of structure-based programming of antibody chemistry has allowed the use of binding energy to traverse reaction coordinates. In this way, unique proteins were obtained that catalyzed many reactions often difficult to achieve by other means [Schultz and Lerner, Science 269, 1835 (1995); Wentworth, Jr. and Janda, Curr. Opin. Chem. Biol. 2, 138 (1998)]. In addition, explanations concerning biological catalysis were elucidated and a substantial body of knowledge acquired regarding the global mechanisms that proteins use to induce and stabilize high-energy species. However, there remains no unified theory that can adequately account for the extraordinary energetics of enzymatic or related protein-cofactor reactions.

The precise interplay between a protein and the high-energy states of a ligand is the essence of biochemical reactivity and catalysis. An increasing appreciation for protein mobility and progress in macromolecular dynamics [Yon, et al., Biochime 80, 33 (1998); Ma, et al., Proc. Natl. Acad.) is augmenting traditional, static “lock-and-key” approaches that include transition-state analogs and other physicochemical interactions aimed at modeling the ground-state reaction coordinate [Radzicka and Wolfenden, Methods Enzymol. 249D, 284 (1995); Mader and Bartlett, Chem. Rev. 97, 1281 (1997)]. However, further insight will require not only a Newtonian analysis of proteins and reaction transition states, but also descriptions in quantum-mechanical terms. Notably, recent theoretical and experimental advances have made it possible to calculate protein wave functions [Roitberg, et al., Science 268, 1319 (1995); Roitberg, et al., J Phys. Chem. B 101, 1700 (1997)] and visualize how vibronic modes between proteins and ligands are coupled during movement across a potential energy surface (Zhu, et al., Science 266, 629 (1994); Wang, et al., Science 266, 422 (1994); Liebl, et al., Nature 401, 181 (1999)]. Therefore, an understanding of catalysis will remain incomplete until the classical and quantum models can be fully integrated.

Because antibodies can translate binding energy along the thermal ground-state surface to lower activation barriers, similar control might also direct the pathways of molecules in high-energy electronically excited states. A ligand that possesses photochemical reactivity as an optical sensor is used to directly report on the interplay between the properties of a protein active site and a chemical event. The present disclosure reports that a series of monoclonal antibodies were prepared against trans-stilbene, a molecule whose excited-state behavior is well understood. Remarkably, even though the antibodies were made to trans-stilbene in its ground-state structure, it was revealed that proteins have an intrinsic capacity to dynamically respond to increases in molecular energy and isolate previously inaccessible quantum states.

### BRIEF SUMMARY OF THE INVENTION

In one aspect, the present invention provides an anti-stilbene antibody. Preferably, the antibody is a monoclonal antibody. Exemplary and preferred such antibodies are designated herein as 19G2, 20F2, 21C6, 22B9, 25F8, 25E2, 23E4, 23G3, 23D3, 23C2, 25C10, 24B6, 21E2, 16H10 and 9E11. The present invention further provides hybridomas that produce and secrete anti-stilbene antibodies.

An antibody of the present invention has particular utility in processes for identifying and/or locating target moieties appended to or incorporating antigenic stilbene. In one embodiment, therefore, the present invention further provides a method of detecting antigenic stilbene. The method includes the steps of exposing antigenic stilbene to an anti-stilbene antibody and detecting an anti-stilbene antibody-stilbene immunoconjugate. Such immunoconjugates can be detected using fluoroscopic procedures.

In a preferred embodiment, the antigenic stilbene is contained in a target moiety. The target moiety can be a protein, a lipid, a carbohydrate or a nucleotide. Preferred target moieties are proteins and nucleotides. Exemplary such target moieties are antibodies and DNA molecules. The present invention further provides target moieties that contain stilbene.

### BRIEF DESCRIPTION OF THE DRAWINGS

In the drawings, which form a portion of the specification

FIG. 1 shows the structure of the stilbene hapten immunogen.

FIG. 2 shows a synthetic scheme for making (E)-4-hydroxyl- and (E)-4-amino-stilbenes.

FIG. 3 shows a synthetic scheme for making (E)-4-iodostilbene.

FIG. 4 shows a synthetic scheme for making 7-{4′-((E)-[2″-phenyl]ethen-1″-yl)phenyl}heptanoic acid methyl ester.

FIG. 5 shows a synthetic scheme for making 11-{4′-((E)-[2″-phenyl]ethen-1″-yl)phenyl}heptanoic acid methyl ester (longer chains).

FIG. 6 shows a synthetic scheme for making intermediates for use with inorganic binders.

FIG. 7 shows a synthetic scheme for making inorganic stilbene binders.

FIG. 8 shows a synthetic scheme for making a diether phospholipid.

FIG. 9 shows a synthetic scheme for making a derivatized phosphonate nucleoside for coupling.

FIG. 10 shows a synthetic scheme for making a derivatized phosphonate nucleoside for coupling.

FIG. 11 shows an illustration of the ground- and excited-state potential energy surfaces for stilbene photochemistry and photophysics. The diagram is the simplest representation of the energy changes for the principal pathways of isomerization and fluorescence in the singlet excited state. Emission from cis-stilbene in fluid solution can only be detected and measured under special conditions, but suggests that the trans and twisted minima are nearly isoenergetic (J. Saltiel, A. S. Waller, D. F. Sears, Jr., J. Am. Chem. Soc. 115, 2453 (1993); J. Saltiel, A. Waller, Y.-P. Sun, D. F. Sears, Jr., J. Am. Chem. Soc. 112, 4580 (1990)).

FIG. 12 shows the panel of EP2 mAbs complexed with 2 and photographed during illumination with UV light. All samples contained 10 mM mAb, except for the background (labeled-mAb), and 20 mM of 2 in a volume of 600 ml PBS (10 mM sodium phosphate, 150 mM NaCl, pH 7.4), 5 percent dimethylformamide (DMF) cosolvent. Samples were prepared in clear, threaded vials made from type 1, class B borosilicate glass (15 mm O.D.×45 mm H; 3.7 ml) (Fisher Scientific) in which the cap closure contained an added teflon liner (Thomas Scientific). The samples were placed in single file on a FisherBiotech (FBTIV-88) variable intensity transilluminator above and along the axis of an internal bulb placement (6 bulbs, 15 W each). The setting was on maximum where the unfiltered output intensity was rated at 1.10 mW/cm² per bulb centered at 312 nm of a 285 nm to 335 nm bandwidth at half-peak height. The photographic exposure conditions were as follows: camera: Nikon N 70; lens: 105 mm macro nikkor; filter: 1A daylight filter (to cut off excess UV light with the minimum effect on color); film: Kodak Ektachrome 64 tungsten (EPY); primary light source: sample fluorescence; secondary light source: halogen modeling lights turned down to be two stops under primary exposure to illuminate labels and caps (labels were later digitally modified); exposure: three seconds@F5.6. Photographs could not capture the color intensity nor distinguish across the range of blue and purple tones, and so did not accurately reproduce what was perceived by the eyes of most observers. Samples 19G2, 20F2, 21C6, and 22B9 were a highly luminous powder-blue color. Sample 25F8 was a less intense, paler blue, and 25E5, 23E4, and 23G3 were similar in intensity to 25F8 but with an added purple hue. The remainder of the antibody samples showed only a purple color to the eye and only 16H10 and 9E 11, with the faintest emissions, could be placed with certainty. The image of the background sample was considerably distorted and to the eye was only a barely perceptible light purple color.

FIG. 13 shows steady-state spectra. (A) UV absorption. (B) Fluorescence excitation. Note: The 19G2-2 complex and free 2 each required a separate y-axis scale for side-by-side comparison of peak shapes. (C) Fluorescence emission. See note for (B). In all cases, see Table 2 for experimental conditions. Steady-state excitation and emission spectra were recorded using an SLM 8100 spectrofluorimeter (Spectronics Instruments) with a bandpass of 4 nm for excitation and emission.

FIG. 14 shows low-temperature transition in blue-fluorescent antibodies. Measurements were made using either 19G2-2 or 20F2-2 complexes with similar results. See Table 2 for experimental conditions.

FIG. 15 shows a view of the stilbene hapten 2 bound to Fab 19G2 (A). Only side-chains within 5 Å of the hapten are shown. The Fₒ-F_{c} electron density map was contoured at 2.0 σ. Gray spheres represent water molecules. FIG. 15(B) shows electrostatic surface map of Fab 19G2. The hapten 2 bound to a relatively uncharged, hydrophobic pocket. (C) A crystal of the 19G2-2 complex under UV irradiation. The crystal was mounted inverted on a depression glass slide and photographed using a Zeiss Axiophot equipped with UV and fluorescence filters. Photographs were taken at 20× magnification with exposure times ranging from 10-60 seconds on Kodak Elakrome ASA400 film.

FIG. 16 shows time-resolved emission decay profiles. Measurements were obtained with picosecond excitation at 318 nm. Decays were measured at 380 nm (free 2, 16H10 and 25E5 complexes) or 410 nm (19G2 complex) by time-correlated single photon counting. Decays were recorded in 4096 channels with a time increment of 18 ps/channel and were normalized relative to the number of counts recorded in the peak channel. See Table 3 for experimental conditions.

FIG. 17 shows reconstructed emission spectra for individual decay components of blue-fluorescent antibodies. The mAb 20F2-2 complex was used as an example. The contribution of decay component i to the total emission intensity at wavelength λ, Iᵢ(λ), was calculated as follows: Iᵢ(λ)=[αᵢ(λ) τᵢ/Σᵢαᵢ(λ) τᵢ]Iₜₒₜ(λ), where τᵢ is the decay time of component i, aᵢ(λ) is the amplitude of component i at wavelength λ, and Iₜₒₜ(λ) is the total steady-state emission intensity at that wavelength. The decay parameters were obtained from multiexponential fits to the intensity decays measured at each wavelength. The spectra for the 92 ps, 1.1 ns and 7.5 ns components were multiplied by a factor of 15 to make them visible on the same vertical axis as the 23 ns component.

FIG. 18 shows the kinetic evolution of the exciplex blue emission. Normalized time-resolved emission profiles of the 20F2-2 complex were recorded at 370 nm (purple) and 480 nm (blue). The solid lines are multiexponential fits to each decay. Emission at 480 nm showed a time-dependent increase with a risetime of 78±10 ps that closely matched the initial decay time of 92±10 ps observed at 370 nm. Decays were recorded in 4096 channels with a time increment of 4.9 ps/channel and were normalized relative to the number of counts recorded in the peak channel. See Table 3 for experimental conditions.

FIG. 19 shows synthesis of a stilbene-tethered C-nucleoside using an amide linker: a) CH₂(OMe)₂, LiBr, TsOH; b) (i) Mg, THF, (ii) 10; c) (i) conc. HCl (cat.), MeOH, 65° C., (ii) PhSO₃H/aq. H₂SO₄ (cat.), toluene, reflux; d) MsCl, NEt₃, CH₂Cl₂; e) NaN₃, DMF, 40° C.; f) PPh₃/H₂O, THF; g) 12, EDC, DMF; h) NaOMe, MeOH; i) glutaric anhydride, DMAP, CH₂Cl₂.

FIG. 20 shows synthesis of a stilbene-tethered C-nucleoside using a polyether linker: a) CH₂(OMe)₂, LiBr, TsOH; b) (i) t-BuLi, THF, −78° C., (ii) 19, (iii) Et₃SiH/BF₃-Et₂O, CH₂Cl₂, −78° C.; c) TMSBr, CH₂Cl₂, −30° C.; d) (i) TfO₂, 2,4,6-collidine, CH₂Cl₂, −70° C., (ii) 23; e) TBAF, THF, 0° C. to rt; f) MsCI, NEt₃, CH₂Cl₂; g) 26, NaH, THF, 60° C.; h) conc. HCl (cat.), MeOH, 65° C.; i) CH₂(OMe)₂, LiBr, TsOH; j) H₂, 10% Pd/C, CHCl₃.

### DETAILED DESCRIPTION OF THE INVENTION

The Invention

The present invention provides anti-stilbene antibodies and their use in identification and localization of target moieties.

Antibodies and Hybridomas

In one aspect, the present invention provides an anti-stilbene antibody. The antibody can be of any class and, thus, includes IgG, IgM, IgA, IgE, and IgD forms. A preferred antibody is a monoclonal antibody. Especially preferred monoclonal antibodies are designated herein as 19G2, 20F2, 21C6, 22B9, 25F8, 25E2, 23E4, 23G3, 23D3, 23C2, 25C10, 24B6, 21E2, 16H10 and 9E11. These antibodies are of the κγ2a or κγ2b isotype, although other isotypes are contemplated herein. Hybridomas that produce and secrete antibodies designated herein as 19G2, 20F2, 21C6, 22B9, 25E5 and 16H10 were deposited at the American Type Culture Collection, 12301 Parklawn Drive, Rockville, Md. on Sep. 11, 2000 and were given ATCC accession numbers PTA-2464, PTA-2465, PTA-2468, PTA-2467, PTA-2466 and PTA-2463, respectively.

The present deposits were made in compliance with the Budapest Treaty requirements that the duration of the deposit should be for 30 years from the date of deposit or for five years after the last request for the deposit at the depository or for the enforceable life of a U.S. patent that matures from this application, whichever is longer. The hybridoma will be replenished should it become non-viable at the depository.

Means for preparing antibodies are well known in the art (Kohler and Milstein, Nature, 256:495, 1975). Monoclonal antibodies are preferred. Monoclonal antibodies are typically obtained from hybridoma tissue cultures or from ascites fluid obtained from mammals into which the hybridoma tissue was introduced.

Conjugates of haptenic stilbene molecules with antigenic (immunogenic) protein carriers such as keyhole limpet hemocyanin (KLH) can be prepared, for example, by activation of the carrier with a coupling agent such as MBS (m-maleimidobenzoyl-N-hydroxy succininide ester), and coupling to the thiol group of the analog-ligand [See, e.g., Liu et al., Biochem., 80, 690 (1979). As is also well known in the art, it is often beneficial to bind a compound to its carrier by means of an intermediate, linking group.

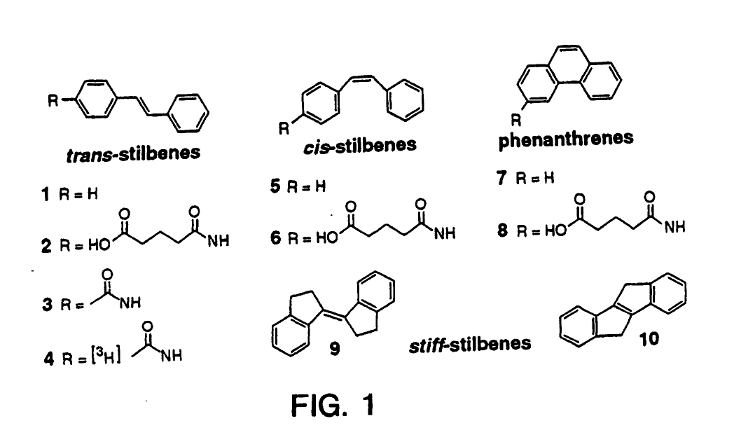

Useful carriers are well known in the art and are generally proteins themselves. Exemplary of such carriers are keyhole limpet hemocyanin (KLH), edestin, thyroglobulin, albumins such as bovine serum albumin or human serum albumin (BSA or HSA, respectively), red blood cells such as sheep erythrocytes (SRBC), tetanus toxoid, cholera toxoid as well as polyamino acids such as poly(D-lysine:D-glutamic acid), and the like. The choice of carrier is more dependent upon the ultimate intended use of the antigen than upon the determinant portion of the antigen, and is based upon criteria not particularly involved in the present invention. For example, if the conjugate is to be used in laboratory animals, a carrier that does not generate an untoward reaction in the particular animal should be selected. In the present study, the immunogen used is shown in FIG. 1. A preferred immunogen is the trans form of stilbene (see FIG. 1).

The carrier-hapten conjugate is dissolved or dispersed in an aqueous composition of a physiologically tolerable diluent such as normal saline, PBS, or sterile water to form an inoculum. An adjuvant such as complete or incomplete Freund's adjuvant or alum can also be included in the inoculum. The inoculum is introduced as by injection into the animal used to raise the antibodies in an amount sufficient to induce antibodies, as is well known.

The foregoing stilbene hapten (FIG. 1) was used to immunize mice and monoclonal antibodies were obtained as described by Niman et al., Proc. Natl. Acad. Sci. USA, 77, 4524 (1980) and Niman et al., in Monoclonal Antibodies and T-Cell Products, Katz, D. H. ed., 23-51, CRC Press, Boca Raton, Fla. (1982). The lymphocytes employed to form the hybridomas of the present invention can be derived from any mammal, such as a primate, rodent (e.g., mouse or rat), rabbit, guinea pig, cow, dog, sheep, pig or the like. As appropriate, the host can be sensitized by injection of the immunogen, in this instance a haptenic analog-ligand, followed by a booster injection, and then isolation of the spleen.

It is preferred that the myeloma cell line be from the same species as the lymphocytes. Therefore, fused hybrids such as mouse-mouse hybrids [Shulman et al., Nature, 276, 269 (1978)] or rat-rat hybrids [Galfre et al., Nature, 277, 131 (1979)] are typically utilized. However, some rat-mouse hybrids have also been successfully used in forming hybridomas [Goding, “Production of Monoclonal Antibodies by Cell Fusion,” in Antibody as a Tool, Marchalonis et al., eds., John Wiley & Sons Ltd., p.273 (1982)]. Suitable myeloma lines for use in the present invention include MPC-11 (ATCC CRL 167), P3×63-Ag8.653 (ATCC CRL 1580), Sp2/0-Ag14 (ATCC CRL 1581), P3X63Ag8U.1 (ATCC CRL 1597), Y3-Ag1.2.3. (deposited at Collection Nationale de Cultures de Microorganisms, Paris, France, number 1-078) and P3X63Ag8 (ATCC TEB 9). The non-secreting murine myeloma line Sp2/0 or Sp2/0-Ag14 is preferred for use in the present invention. The hybridoma cells that are ultimately produced can be cultured following usual in vitro tissue culture techniques for such cells as are well known. More preferably, the hybridoma cells are cultured in animals using similarly well-known techniques with the monoclonal receptors being obtained from the ascites fluid so generated.

In particular, an exemplary monoclonal receptor was produced by the standard hybridoma technology of Kohler et al., Nature, 256, 495 (1975) and Engvall, E., Methods Enzymol., 70, 419 (1980). Specifically, female mice were immunized by intraperitoneal injection with an inoculum of stilbene hapten in of a 1:1 mixture of phosphate buffered saline (PBS), pH 7.4, and complete Freund's adjuvant. Two weeks later, the mice were again injected in a like manner. After an additional eight weeks, the mice were immunized intravenously with hapten in PBS (pH 7.4). The spleens were removed from the mice four days later, and the spleen cells were fused to myeloma cells.

The spleen cells were pooled and a single cell suspension was made. Nucleated spleen cells were then fused with Sp2/0-Ag14 non-secreting myeloma cells in the presence of a cell fusion promoter (polyethylene glycol 2000). A hybridoma that produces a particular monoclonal antibody was selected by seeding the spleen cells in 96-well plates and by growth in Dulbecco's modified Eagle medium (DMEM) containing 4500 mg/liter glucose (10 percent), 10 percent fetal calf serum (FCA), hypoxanthine, aminopterin and thymidine (i.e., HAT medium) which does not support growth of the unfused myeloma cells.

After two to three weeks, the supernatant above the cell clone in each well was sampled and tested for the presence of antibodies against stilbene. Positive wells were cloned twice by limiting dilution. Those clones that continued to produce stilbene-specific antibody after two clonings were expanded to produce larger volumes of supernatant fluid. The hybridoma and the monoclonal receptors produced therefrom and described herein are identified by the laboratory designation as discussed hereinbefore.

A monoclonal receptor of the present invention can also be produced by introducing, as by injection, the hybridoma into the peritoneal cavity of a mammal such as a mouse. Preferably, as already noted, syngeneic or semi-syngeneic mammals are used, as in U.S. Pat. No. 4,361,549, the disclosure of which is incorporated herein by reference. The introduction of the hybridoma causes formation of antibody-producing hybridomas after a suitable period of growth, e.g., 1-2 weeks, and results in a high concentration of the receptor being produced that can be recovered from the bloodstream and peritoneal exudate (ascites) of the host mouse. Although the host mice also have normal receptors in their blood and ascites, the concentration of normal receptors is typically only about five percent that of the monoclonal receptor concentration. Monoclonal receptors are precipitated from the ascitic fluids, purified by anion exchange chromatography, and dialyzed against three different buffers.

The abundance of acetyl and butyl cholinesterase in red blood cells and serum [Stedman et al., Biochem. J. 26: 2056 (1932); Alles et al., Biol Chem., 133: 375 (1940)] necessitated extra caution during purification of the antibody molecules. In the present study, IgG molecules were typically obtained from mouse ascites fluid via anion-exchange chromatography on a DEAE Sepharose column followed by affinity chromatography on a Protein G Sepharose column and then again by anion exchange chromatography on a Mono Q column. As a control, authentic acetyl and butyl cholinesterases were not retained in the affinity column when fractionated under the same conditions employed for antibody purification.

Antibodies obtained were judged to be greater than 98 percent homogeneous by sodium dodecyl sulfate polyacrylamide gel electrophoresis [Laemmli, V. Nature, 227: 680 (1970)]. The resulting concentrated solutions containing isolated IgG fractions were typically prepared into stock solutions of antibody at 1-20 mg/ml using an appropriate buffer such as 50 mM Tris-HCl or sodium phosphate containing 0.01 M sodium azide.

Uses

An anti-stilbene antibody of this invention has a variety of uses. Those uses relate to the unique fluorescent characteristics of immune complexes formed between the antibody and stilbene. Excitation of the complex with long-wavelength ultraviolet light having a wavelength of from about 300 nm to about 350 run results in fluorescent emission of light ranging from light purple (having a maximum of about 380 nm) to deep blue (having a maximum of about 410 nm). Thus, when excited with a particular light, the complex emits light within the blue spectrum. Measurement of this blue emitted light allows for localization, identification and quantification of immune complex formation. Neither the antibody alone nor stilbene alone emits blue light when excited.

Fluorescence is the luminescence of a substance from a single electronically excited state, which is of very short duration after removal of the source of radiation. The wavelength of the emitted fluorescence light is longer than that of the exciting illumination (Stokes' Law), because part of the exciting light is converted into heat by the fluorescent molecule.

Detection of the emitted fluorescent light from a stilbene/anti-stilbene antibody conjugate of this invention can occur in a wide variety of media including, but not limited to, gels, culture media, physiological fluids (e.g., blood, serum, plasma, urine) and the like. Detection of fluorescence can be detected where the conjugate is situated in vitro, in situ or in vivo [See, e.g., U.S. Pat. No. 5,650,135, the disclosure of which is incorporated herein by reference; Sweeney et al., Proc. Natl. Acad. Sci., 96(21):12044-12049, (1999); Edinger et al., Neoplasia, 1(4):303-310, (1999); Contag et al., Photochem. And Photobiol., 66(4):529-531 (1997); Benaron et al., Phil. Trans. R. Soc. Lond. B, 352:755-761 (199); Contag et al., Mol. Microbiol., 18(4):593-603, (1995); and Contag et al., Biomed. Optical Spect. Diag., 3:220-224 (1996)].

When used with human subjects, precautions are typically taken to shield the excitatory light so as not to contaminate the fluorescence photon signal being detected. Obvious precautions include the placement of an excitation filter, such that employed in fluorescence microscope, at the radiation source. An appropriately-selected excitation filter blocks the majority of photons having a wavelength similar to that of the photons emitted by the fluorescent moiety. Similarly a barrier filter is employed at the detector to screen out most of the photons having wavelengths other than that of the fluorescence photons. Filters such as those described above can be obtained from a variety of commercial sources, including Omega Optical, Inc. (Brattleboro, Vt.).

Alternatively, a laser producing high intensity light near the appropriate excitation wavelength, but not near the fluorescence emission wavelength, can be used to excite the fluorescent moieties. As an additional precaution, the radiation source can be placed behind the subject and shielded, such that the only radiation photons reaching the site of the detector are those that pass all the way through the subject. Furthermore, detectors may be selected that have a reduced sensitivity to wavelengths of light used to excite the fluorescent moiety.

An anti-stilbene antibody can be used to detect the presence of any moiety that contains antigenic stilbene. As used herein, the phrase “antigenic stilbene” means stilbene, whether alone or attached to or complexed with another moiety, which stilbene forms an immune complex with a present antibody. The moiety can take the form of, for example, molecules, macromolecules, particles, microorganisms, or cells. The methods used to conjugate stilbene to a moiety depend, as is well known in the art, on the nature of the moiety. Exemplary conjugation methods are discussed in the context of the moieties described below.

Small molecule moieties that may be useful in the practice of the present invention include compounds which specifically interact with a endogenous ligand or receptor. Examples of such moieties include, but are not limited to, drugs or therapeutic compounds, hormones, growth factors, cytokines, bioactive peptides and the like.

The small molecules are preferably conjugated to stilbene by any of a variety of methods known to those skilled in the art. The small molecule moiety can be synthesized to contain a stilbene, so that no formal conjugation procedure is necessary or synthesized with a reactive group that can react with stilbene. Small molecules conjugated to stilbene can be used either in animal models of human conditions or diseases, or directly in human subjects to be treated. For example, a small molecule which binds with high affinity to receptor expressed on tumor cells may be used in an animal model to localize and obtain size estimates of tumors, and to monitor changes in tumor growth or metastasis following treatment with a putative therapeutic agent.

Macromolecules, such as polymers and biopolymers, constitute another example of moieties useful in practicing the present invention. Exemplary macromolecules include antibodies, antibody fragments, proteins, fusion proteins and nucleotides. Bifunctional antibodies or antibody fragments can be used to localize their antigen in a subject by conjugating the antibodies to stilbene, administering the conjugate to a subject by, for example, injection, allowing the conjugate to localize to the site of the antigen, and imaging the conjugate. Particles, including beads, liposomes and the like, constitute another moiety useful in the practice of the present invention. Due to their larger size, particles can be conjugated with a larger number of stilbene molecules than, for example, can small molecules. This results in a higher concentration of stilbene, which can be detected using shorter exposures or through thicker layers of tissue. In addition, liposomes can be constructed to contain an essentially pure targeting moiety, or ligand, such as an antigen or an antibody, on their surface. Further, the liposomes may be loaded with relatively high concentrations of stilbene.

In one embodiment, the present invention includes a method for detecting the localization of a target moiety in a mammalian subject. The method includes administering to the subject a conjugate of the entity and stilbene. The moiety may be conjugated to stilbene by a variety of techniques, including incorporation during synthesis of the moiety, chemical coupling post-synthesis, or non-covalent association. After a period of time in which the conjugate can localize in the subject, the subject is immobilized within the detection field of a photodetector device for a period of time effective to measure a sufficient amount of light emission to construct an image. The method described above can be used to track the localization of the moiety in the subject over time, by repeating the imaging steps at selected intervals and constructing images corresponding to each of those intervals.

The target moiety may be an inherent property of the entity. Examples of target moieties include antibodies, antibody fragments, enzyme inhibitors, receptor-binding molecules, various toxins and the like. Targets of the target moiety may include sites of inflammation, infection, thrombotic plaques and tumor cells. Markers distinguishing these targets, suitable for recognition by targeting moieties, are well known.

In a related embodiment, the invention includes a method for detecting the level of a biocompatible moiety in a subject over time. The method is similar to methods described above, but is designed to detect changes in the level of the moiety in the subject over time, without necessarily localizing the moiety in the form of an image. This method is particularly useful for monitoring the effects of a therapeutic substance, such an antibiotic, on the levels of a target moiety. In another aspect the invention includes a method of identifying therapeutic compounds effective to inhibit spread of infection by a pathogen. The method includes administering a conjugate of the pathogen and stilbene to control and experimental animals, treating the experimental animals with a putative therapeutic compound, localizing the pathogen in both control and experimental animals by the methods described above, and identifying the compound as therapeutic if the compound is effective to significantly inhibit the spread or replication of the pathogen in the experimental animals relative to control animals.

In still another aspect, the invention includes a method of localizing moieties conjugated to stilbene through media of varying opacity. The method includes the use of a photodetector device to detect light emitted and transmitted through the medium, integrate the light over time, and generate an image based on the integrated signal. An exemplary media for use with this method is a gel such as used for the separation of proteins and nucleic acids.

In yet another embodiment the invention includes a method of measuring the concentration of selected target moieties at specific sites in an organism. The moiety containing stilbene is administered such that it adopts a substantially uniform distribution in the animal or in a specific tissue or organ system (e.g., spleen). The organism is imaged, and the intensity and localization of light emission is correlated to the concentration and location of the target moiety.

In another aspect, the invention includes a method of identifying therapeutic compounds effective to inhibit the growth and/or the metastatic spread of a tumor. The method includes (i) administering tumor cells labeled with or containing stilbene to groups of experimental and control animals, (ii) treating the experimental group with a selected compound and with an anti-stilbene antibody, (iii) localizing the tumor cells in animals from both groups by imaging light emission from the tumor cells with a photodetector device, and (iv) identifying a compound as therapeutic if the compound is able to significantly inhibit the growth and/or metastatic spread of the tumor in the experimental group relative to the control group.

In another aspect, this invention provides target moieties that contain stilbene. Exemplary and preferred moieties are nucleosides, nucleotides and nucleic acids (RNA, DNA). The following schemes show the synthesis (E)-stilbene derivatives which can be bonded to molecules of biological interest. These derivatives can be used to functionalize all of the title biological molecules. The method of functionalization of some of the biological molecules is well known in the art and will not be described here.

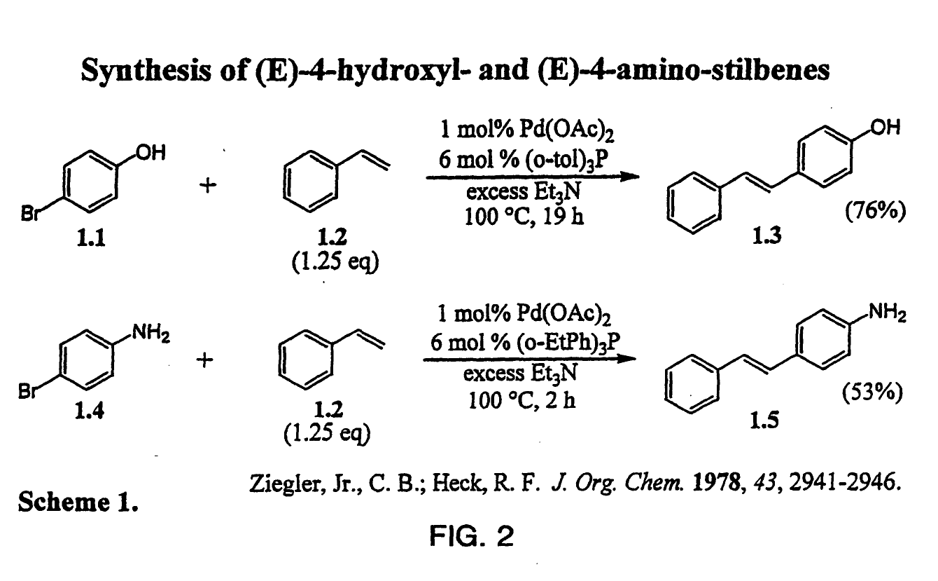

The first scheme (Scheme 1, FIG. 2) shows the synthesis of two important stilbene derivatives, 1.3 and 1.5, that can be modified as shown in later schemes. A Heck reaction between the unprotected p-bromophenol (1.1) or p-bromoaniline (1.4) and styrene (1.2) using palladium (II) acetate as a catalyst gives the desired products in good yield. All of these starting materials are currently available from Aldrich.

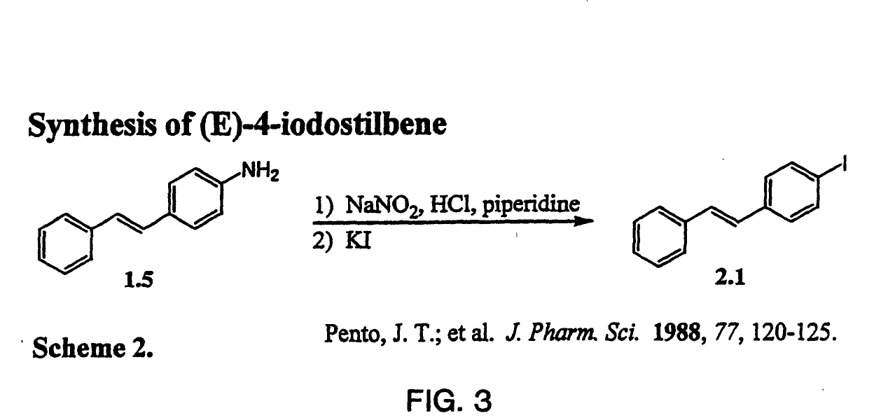

The (E)-4-iodostilbene (2.1) is made by diazotization of the aniline derivative (1.5) and reaction with iodide (Scheme 2, FIG. 3). This iodide is used in the cross-coupling reactions forming the alkyl derivatives of (E)-stilbene.

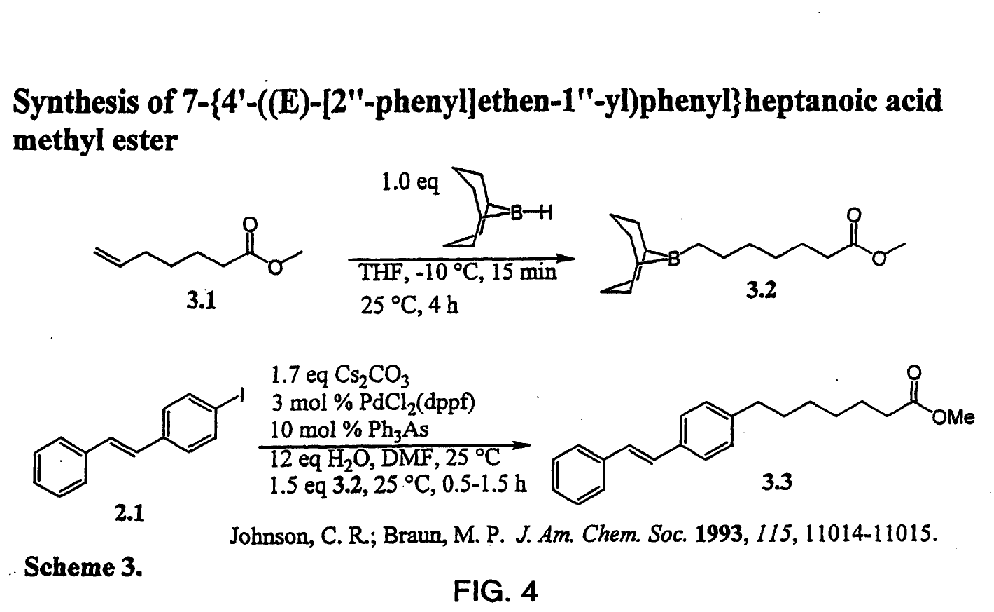

As shown in Scheme 3 (FIG. 4) a terminally functionalized alkyl derivatives can be obtained from a Suzuki reaction between the alkyl-9-BBN (3.2) and the iodostilbene (2.1). The ω-alkene methyl ester (3.1) is cleanly hydroborated with 9-BBN in THF and this derivative (3.2) is used in the coupling reaction directly without isolation. The reactions of Scheme 3 can be applied to any length of ω-alkene methyl ester to obtain various tether lengths between the biological molecules and the stilbene moiety.

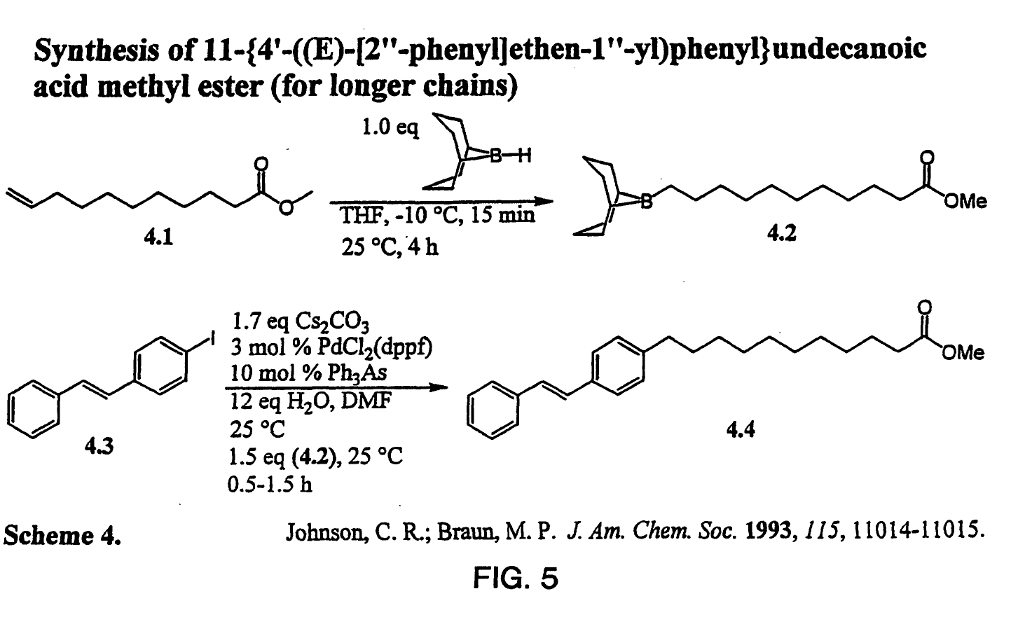

Scheme 4 (FIG. 5) shows the methyl ester of 10-undecenoic acid (4.1) used in the same manner. These particular substrates are shown because the ω-alkene acids are commercially available and the methyl esters are synthesized by treatment with diazomethane. The methyl esters are saponified to the free acids for use in making amides or esters with amine-containing or hydroxyl-containing biological molecules. The saponification is best done with two equivalents of lithium hydroxide in THF and water at room temperature.

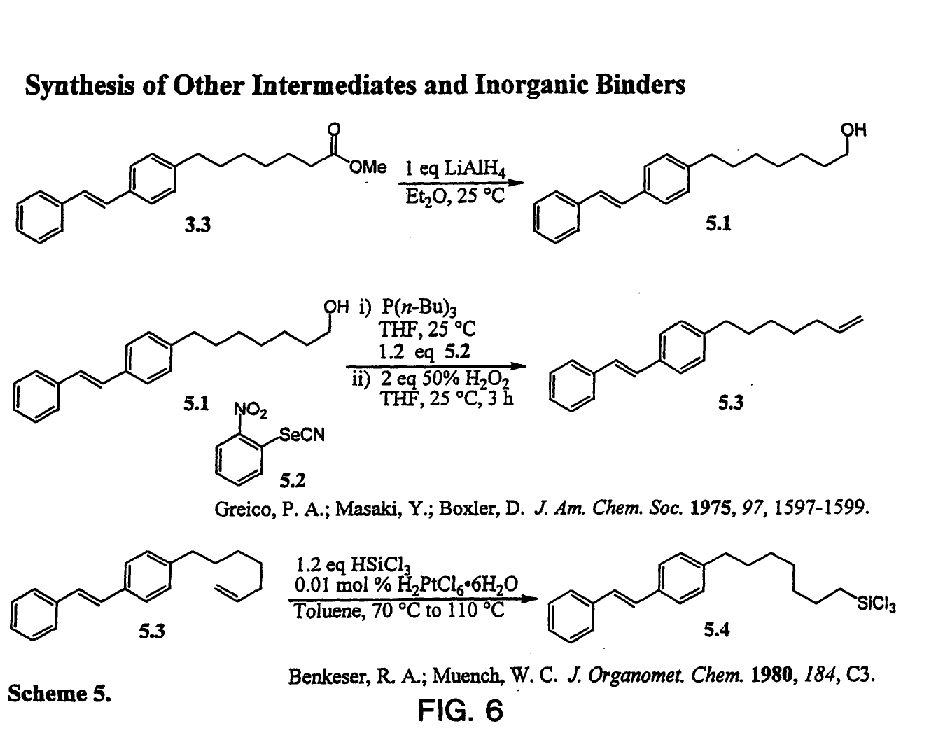

Scheme 5 (FIG. 6) shows the ester (3.3) being reduced to the alcohol (5.1) to give a substrate that is useful for forming acetals with carbohydrates or monosaccharides. The alcohols are easily converted to the terminal olefins by treatment with tri-n-butylphosphine and o-nitrophenyl selenocyanate (5.2) followed by reaction with two equivalents of 50% hydrogen peroxide in TBF. The selenoxide that is formed quickly undergoes elimination to give the terminal olefin. The olefin (5.3) can be a substrate for another hydroboration-Suzuki coupling to a vinyl or aryl bromide or iodide. Alternatively, it can be cleaved with ozone and the aldehyde can be used in a reductive amination reaction with an α,ω-diamine to give a diamine that has a primary and secondary amine.

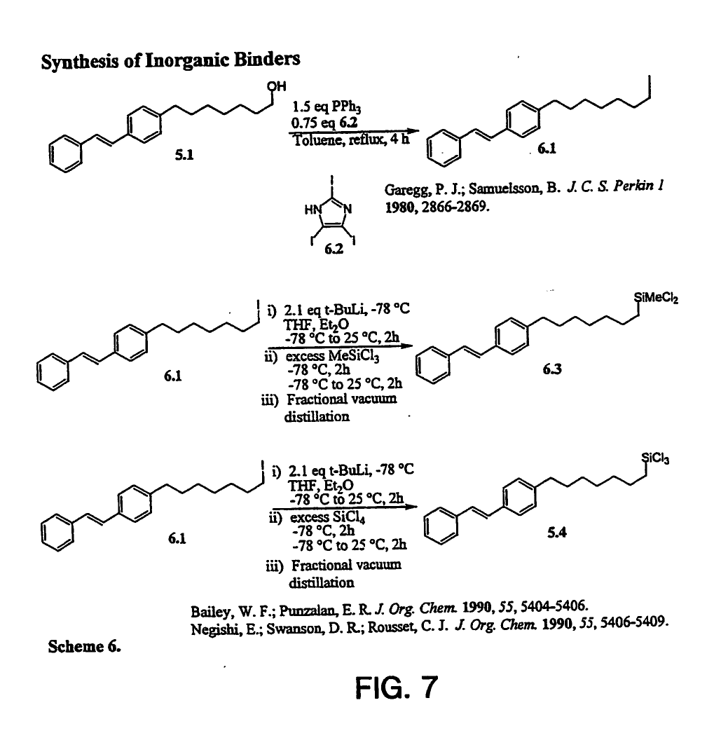

Binding to inorganic substrates through a thiol or a siliconate group is known. A thiol is obtained from an alcohol by a Mitsunobu reaction or by displacement of the corresponding iodide with an excess of sodium sulfide. Thiols are known to bind to flat gold surfaces as a monolayer. Glass or metal oxides are derivatized by monoalkylsilyl or dialkylsilyl chlorides in the presence of an amine base. The synthesis of exemplary dichlorosilanes and trichlorosilanes (5.4) is shown using two methods. The first method is by hydrosilylation of a terminal olefin (5.3) with trichlorosilane in the presence of a small amount of hydrogen hexachloroplatinate (IV) hydrate in toluene (Scheme 5, FIG. 6). The second method uses the alkyl iodide (6.1) synthesized from the alcohol 5.1, which is first converted to the alkyllithium and then reacted with an excess of alkyltrichlorosilane or tetrachlorosilane (Scheme 6, FIG. 7). The products, 6.3 and 5.4, can be recovered by fractional vacuum distillation.

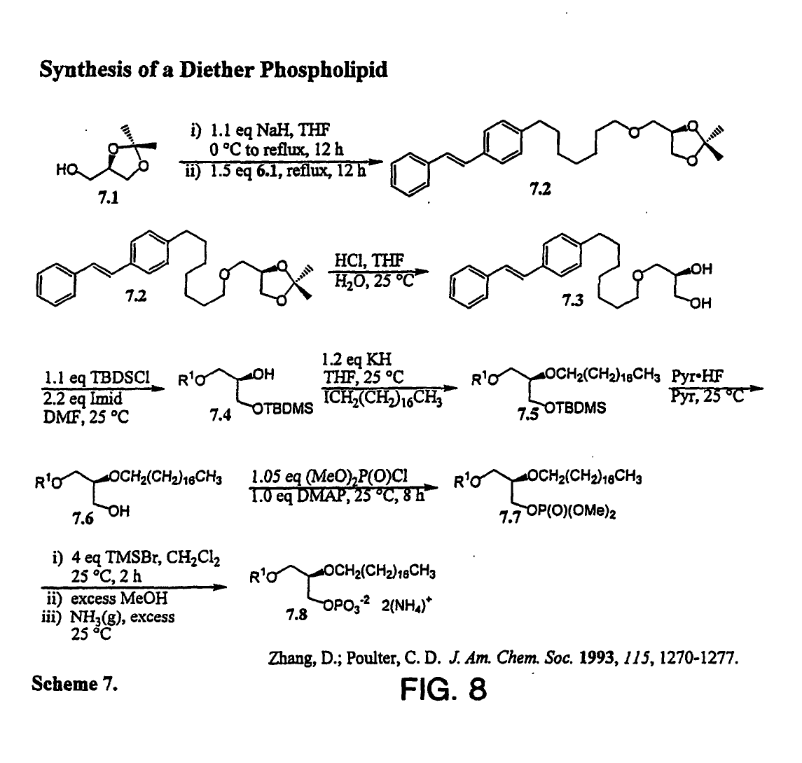

Analogs for phosphoglycerides, glycerides, phosphiditates or ether phospholipids are easily synthesized from the above stilbene derivatives by methods known in this art. An example of the synthesis of an ether phospholipid is shown in Scheme 7 (FIG. 8), where a glycerol derivative, 7.1, is alkylated by an alkyl iodide derivative of stilbene (6.1). The ether (7.2) is deprotected to reveal the diol (7.3) and after selective protection of one of the hydroxyl groups one obtains 7.4. Alkylation with another iodide gives the diether 7.5. Deprotection of the alcohol with pyridine-hydrofluoric acid in pyridine gives the primary alcohol 7.6 which is then phosphorylated. The phosphate triester (7.7) is hydrolyzed selectively and the phosphoryl dibromide intermediate is hydrolyzed to the phosphate salt 7.8.

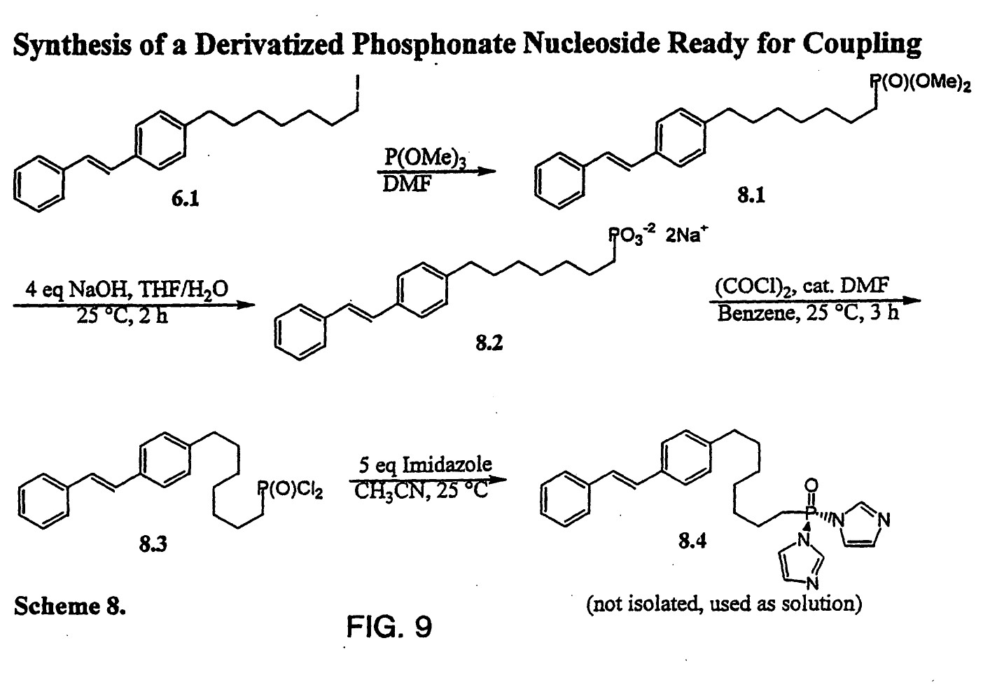

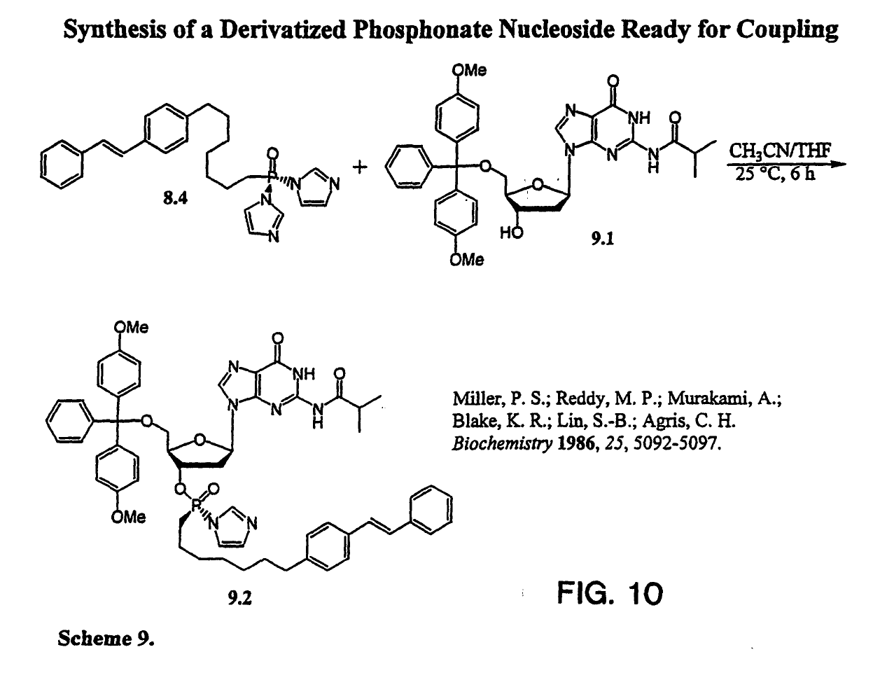

The easiest way to derivatize DNA or RNA is to incorporate a phosphonate group into the sugar-phosphate backbone. This allows the derivative to be made as a separate subunit which can be incorporated into the synthesized single strand DNA or RNA. The synthesis of a deoxyguanosine phosphonate derivative is shown in Schemes 8 (FIG. 9) and 9 (FIG. 10). The product, 9.2, is suitable for use in the phosphotriester or the phosphite triester solid phase syntheses. A single example is given here and making derivatives of phosphonates analogs is well known to those who are skilled in the art.

Example 2, below, provides detailed schemes for making nucleosides that contain stilbene. The Examples that follow illustrate preferred embodiments of the present invention and are not limiting of the specification and claims in any way.

### EXAMPLE 1

### Antstilbene Antibodies

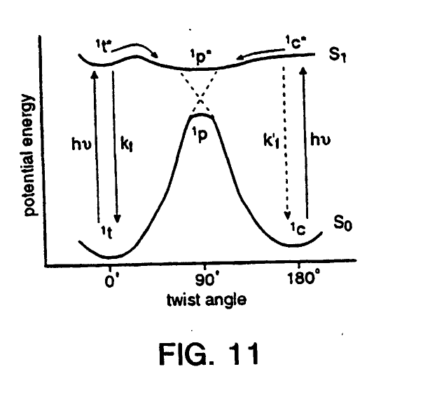

Principle and design. The photophysics and photochemistry of trans-stilbene 1 has been extensively investigated (J. Saltiel and Y.-P. Sun, in Plotochromism: molecules and systems, H. Dürr, H. Bouas-Laurent, Eds. (Elsevier, N.Y., 1990), pp. 64-162; D. H. Waldeck, Chem. Rev. 91, 415 (1991); H. Gorner and H. J. Kuhn, Adv. Photochem. 19, 1 (1995)). Two decay processes, fluorescence and isomerization to cis-stilbene 5, can account for the excited-state behavior of 1 in solution (FIG. 1, FIG. 11). Notably, the isomerization pathway is the predominant funnel for quenching of fluorescence at room temperature. The singlet mechanism for the transπcis photoisomerization was proposed by Saltiel (J. Saltiel, J. Am. Chem. Soc. 89, 1036 (1967)) and was validated through comprehensive singlet and triplet quenching studies (H. Görner and H. J. Kuhn, Adv. Photochem. 19, 1 (1995)). The fundamental model suggests that after excitation of the trans form to the excited trans-singlet state (¹t*) twisting about the carbon-carbon double bond converts the molecule into the excited perpendicular singlet state (¹p*). Subsequently, internal conversion to the perpendicular ground state (¹p) followed by rotational relaxation to the cis and trans ground states completes the process. The transπcis photoisomerization occurs from an angle of twist of 0° to 90° by rotating in S_{I} and from 90° to 180° in the So state. The reverse occurs for the cisπtrans reaction (FIG. 11).

In order to study the excited-state behavior of a traits-stilbene molecule at an antibody combining site, it was necessary to obtain monoclonal antibodies (mAbs) using an appropriate stilbene hapten. Although it should be possible to elicit mAbs highly specific for the parent trans-stilbene 1, we desired a derivative more suitable for experiments in aqueous media. Hence, the design required a functional group on 1 that would 1) afford coupling to a carrier protein, 2) enhance water solubility, 3) be stable during routine irradiation, and 4) introduce minimal alteration of the electronic nature of 1. The latter was considered important so that the vast body of information available for trans-stilbene itself would be applicable to a substituted analog. A glutaric amide was considered an ideal candidate and resulted in the preparation of hapten 2 (FIG. 1). The compound 2 was prepared as follows. Benzyltriphenylphosphonium bromide (14.3 g, 33 mmol) was slurried in tetrahydrofuran (THF) (165 ml) and cooled to 0° C. under nitrogen. Butyllithium (13.9 ml, 34.5 mmol; 1.6 M in hexane) was added and the mixture stirred at 0° C. for 15 min, room temperature for 1 h, and then cooled again to 0° C. A solution of 4-nitrobenzaldehyde (5 g, 33 mmol) in THF was added and the mixture stirred at room temperature for 18 h. The reaction was quenched with 5% citric acid, extracted with ethyl acetate (EtOAc), washed with water, brine, dried over sodium sulfate and evaporated to a yellow solid. The solid was triturated three times with hexane/EtOAc (90/10) (100 ml) and the liquor decanted from the solid each time. The solid was the pure trans-4-nitrostilbene (1.6 g). The liquor was concentrated to 100 ml, filtered, and the filtrate evaporated to a yellowish oil of 4-nitrostilbene that was a mixture trans and cis isomers (1/8.5) (5 g). A solution of trans-4-nitrostilbene (1.6 g, 7.1 mmol) was slurried in ethanol (20 ml) and reduced with SnCl₂—H₂O (8 g, 36 mmol) at 70-75° C. for 1 h under nitrogen. The mixture was poured into EtOAc (500 ml), washed with water, saturated sodium bicarbonate, dried over sodium sulfate, filtered and evaporated. The residue was purified using flash chromatography (hexane/EtOAc=70/30) that afforded the trans-4-aminostilbene as a tan solid (1.4 g, 99%). A solution of trans-4-aminostilbene (0.53 g, 2.7 mmol) in dichloromethane (CH₂Cl₂) (10 ml) was stirred at room temperature and triethylamine (1.13 ml, 7.1 mmol) was added followed by glutaric anhydride (464 mg, 4.05 mmol) and 4-dimethylaminopyridine (DMAP) (2 mg). The solution was stirred at room temperature for 18 h, poured into water/EtOAc, shaken, filtered and the solid washed with water, EtOAc, hexane, and then dried that afforded 2 as a white solid (340 mg, 41%). The Hammett σ value provided a measure of the degree to which substituents perturbed the electronic nature of an aromatic ring (O. Exner, Correlation Analysis of Chemical Data (Plenum Press, New York, 1988)). For the related acetamido group σₚ=−0.01, close to the zero value assigned to hydrogen and suggested 2 would be electronically comparable to 1. Immunization with a keyhole limpet hemocyanin (KLH) conjugate of 2 resulted in a panel of 15 mAbs for analysis (G. Këohler and C. M. Milstein, Nature 256, 495 (1975). The hybridomas were derived from fusions with an X63-AG8.653 myeloma cell line. All mAbs were purified from ascites to ˜95% homogeneity as follows: step 1, saturated ammonium sulfate; step 2, DEAE Sephacel (Pharmacia) chromatography; step 3, protein G affinity chromatography).

The fluorescence quantum yield (φ_{f}) of flexible molecules that can undergo facile torsional displacements in the excited-singlet state increases greatly in high viscosity or low-temperature, rigid media. Hence, for 1 there is an increase in fluorescence efficiency with a concurrent decrease in the efficiency of transπcis photoisomerization (J. Saltiel, J. T. D'Agostino, J. Am. Chem. Soc. 94, 6445 (1972); S. Malkin, E. Fischer, J Phys. Chem. 68, 1153 (1964)). Similarly, when substitutions are made that constrain or “stiffen” the structure of 1 from twisting as in 10, the isomerization yield is zero and the φ_{f} approaches unity (J. Saltiel, A. Marinari, D. W. L. Chang, J. C. Mitchener, E. D. Megarity, J. Am. Chem. Soc. 101, 2982 (1979); J. Saltiel, O. C. Zafiriou, E. D. Megarity, A. A. Lamola, J. Am. Chem. Soc. 90,4759 (1968)). Consequently, attributing the characteristic of “stiffness” to 2 within the confines of a specific antibody binding site suggested a reduction in isomerization and enhanced φ_{f}. However, the outcome could not be predicted and was quite unexpected.

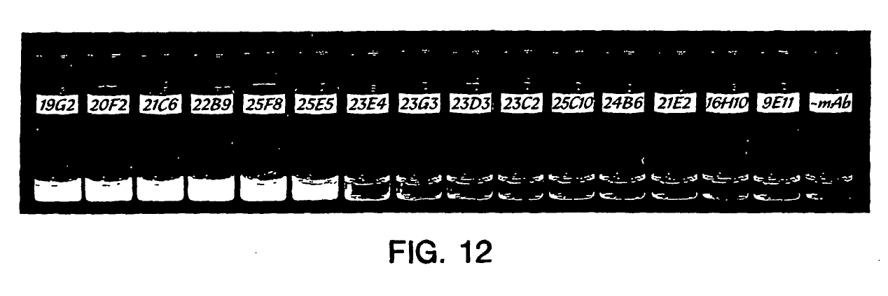

Initial observations. When the mAbs were each mixed with 2 in stoichiometric amounts and irradiated with ultraviolet (UV) light, a surprising phenomenon was observed. Several antibodies, 19G2, 20F2, 21C6, and 22B9 immediately produced an intense, powder-blue colored fluorescence (FIG. 12). Moreover, the panel of mAbs revealed a range of visually discernable colors and/or intensities over the purple (violet) to blue region of the spectrum. A similar result was found with 1 although not as dramatic since comparable concentrations were not possible. The background reaction (−mAb) of 2 alone showed a very faint purple fluorescence typical of that for trans-stilbene in solution at room temperature. Antibodies themselves afforded no observable fluorescence. In light of the programmed specificity of the antibodies for 2, it was concluded that mAb-2 complexes were the source of the emitted light.

The distinct fluorescence of a blue-emitting antibody such as 19G2 was extremely robust in that there was no visible effect upon saturation with oxygen, variation of the pH from 4 to 11, a change in temperature from −5 to 50° C., or prolonged irradiation. Complete photobleaching to a colorless and turbid solution occurred only after 60 minutes of continuous UV exposure under the conditions described (FIG. 12). Yet, remarkably, freezing a solution of a blue-antibody complex in either a dry ice-isopropanol bath (sample temperature −60° C., 213° K) or liquid nitrogen bath (sample temperature −179° C., 94° K) followed by UV irradiation resulted in the complete absence of the blue fluorescence that reappeared upon thawing. In the frozen state in buffer solution, the 19G2-2 complex was a semi-translucent frozen solution in which the emission appeared as a purple color. This was similar to a frozen sample of the stilbene 2 alone, or a room temperature or frozen complex of a typical purple-emitting mAb such as 16H10. The loss of blue color was counterintuitive given the fact that fluorescent behavior of molecules was generally enhanced at low temperatures.

A number of hypotheses for the origin of the unusual blue fluorescence were considered (J. Saltiel, O. C. Zafiriou, E. D. Megarity, A. A. Lamola, J. Am. Chem. Soc. 90, 4759 (1968)). At this stage, not yet having obtained structural data for an antibody-hapten complex, we conjectured that exciplex-like interactions of 2 at the combining site could be responsible. However, given the observed temperature-dependent phenomenon, it was apparent that simple static interactions between stilbene and antibody were not sufficient to produce the emission of blue light. We suspected that dynamic events were operative and sought further evidence to support this view.

Steady-state analysis: spectroscopy and energetics. Determination of K_{d} values for the trans-stilbene 2 immediately revealed no extraordinary tight-binding effects in the ground state and no substantial differences between the unique blue-fluorescent mAbs and the majority of the other antibodies in the panel (Table 1).

TABLE 1
Steady-state thermodynamic parameters for EP2 mAbs.*

| EP2 | IgG | K_{d, trans}† | K_{d,cis}‡ | [trans/cis]ₚₛₛ§ |
|---|---|---|---|---|
| mAb | isotype | (μM) | (μM) | (%) |
| 19G2 | κγ2b | 0.16 | 1.7 | 97/3 |
| 20F2 | κγ2b | 0.30 | 3.8 | 97/3 |
| 21C6 | κγ2b | 0.20 | 1.6 | 95/5 |
| 22B9 | κγ2a | 0.25 | 1.6 | 93/7 |
| 25F8 | κγ2a | 0.53 | 3.3 | 95/5 |
| 25E5 | κγ2a | 0.18 | 1.4 | 96/4 |
| 23E4 | κγ2b | 1.0 | 0.60 | 83/17 |
| 23G3 | κγ2b | 0.87 | 5.1 | 81/19 |
| 23D3 | κγ2b | 0.60 | 3.2 | 67/33 |
| 23C2 | κγ2a | 0.24 | 1.7 | 66/34 |
| 25C10 | κγ2a | 0.26 | 1.8 | 69/31 |
| 24B6 | κγ2a | 0.26 | 2.0 | 93/7 |
| 21E2 | κγ2a | 0.26 | 2.2 | 68/32 |
| 16H10 | κγ2a | 0.31 | 2.3 | 69/31 |
| 9E11 | κγ2a | 0.20 | 0.10 | 80/20 |
| *All reactions were conducted in PBS (10 mM sodium phosphate, 150 mM NaCl, pH 7.4), 5% DMF cosolvent, at 21° C. The mAbs were arranged in the order originally established by visual observation (FIG. 12). All stock and reaction solutions containing 2 and 6 were protected from light using foil when not in use. Stock solutions of 2 and 6 were prepared in DMF and the former contained 0.50% of 6 and the latter contained 0.28% of 2. †Determined directly for 4 by equilibrium dialysis. | | | | |
| # The K_{d} values for 2 were approximately the same (±10%) as measured by competition equilibrium dialysis versus 4. ‡Determined by competition equilibrium dialysis of 6 versus 4. All K_{d} values were accurate to ±10%. §Photostationary state (pss) reactions were carried out using the glass vials and UV illuminator (FIG. 12). The concentrations were 60 μM mAb and 20 μM of 2 or 6 each of which afforded the | | | | |
| # same pss (±1% for each isomer). The values from 2 were tabulated. Reaction times were 30 s in order to observe complete equilibration up to the point of trace (<1-1%) formation of 8 in some cases. The pss was usually achieved in 10-25 s depending on the mAb. The pss for the background reaction (no mAb) was 28/72 after 5-6 s. The amounts of 2 and 6 were determined using reversed-phase HPLC (C-18 column, VYDAC 201TP54; isocratic mobile phase of 39% acetonitrile, 61% water (0.1% | | | | |
| # trifluoroacetic acid); flow rate = 1.6 ml/min; detector setting = 300 nm; retention times: 8 = 6.20 min, 6 = 7.24 min, 2 = 8.28 min, benzophenone standard = 10.46 min; relative peak heights 2:6:8 were 1.2:1.1:1.0, and minimum detection limits were 0.25 μM, 0.20 μM, 0.20 μM, respectively). | | | | |

In fact, although some purple antibodies were among those with the worst affinities, the most weakly purple-fluorescent mAbs 9E11 and 16H10 had a K_{d} comparable to the blue-emitting antibodies. However, a striking contrast was observed in the φ_{f} values as well as differences in the absorption, excitation, and emission spectra of the blue-fluorescent antibody complexes (Table 2).

TABLE 2
Steady-state spectral data for mAb complexes and stilbenes.*

| EP2 | λem | λex | UV absorption | εₘₐₓX 10⁻⁴ | φf |
|---|---|---|---|---|---|
| mAb | (nm) | (nm) | bands (nm)† | (M⁻¹ cm⁻¹) | ‡ |
| 19G2 | 410 | 327, 340 | (310), 325, (340) | 3.15 | 0.78 |
| 20F2 | 410 | 327, 340 | (310), 325, (340) | 3.15 | 0.80 |
| 22B9 | 410 | 327, 340 | (310), 325, (340) | 3.00 | 0.69 |
| 21C6 | 410 | 327, 340 | (310), 325, (340) | 3.18 | 0.64 |
| 25F8 | 408 | 332, 346 | 332, (349) | 3.22 | 0.62 |
| 25E5 | 387 | 334, 347 | 332, (349) | 3.32 | 0.63 |
| 23E4 | 399 | 328, 339 | 336, (352) | 3.09 | 0.55 |
| 23G3 | 390 | 337, 351 | 336, (352) | 3.09 | 0.57 |
| 23D3 | 380 | 328 | 323 | 2.82 | 0.31 |
| 23C2 | 382 | 327 | 320 | 2.85 | 0.25 |
| 25C10 | 380 | 328 | 322 | 2.91 | 0.27 |
| 24B6 | 381 | 332, 345 | 328, (346) | 2.97 | 0.46 |
| 21E2 | 380 | 327 | 336, (352) | 3.09 | 0.57 |
| 16H10 | 380 | 327 | 320 | 2.82 | 0.28 |
| 9E11 | 387 | 327 | 320 | 3.03 | 0.17 |
| 2 | 388 | 325 | 320 | 3.32 | 0.02 |
| 9 | 442 | 336, 353 | 340, (361) | 3.00 | nd§ |
| 10 | 362∥ | 317,330 | (310), 323; (344) | 2.22 | nd§ |
| *Unless otherwise noted, all measurements were made in PBS (10 mM sodium phosphate, 150 mM NaCl, pH 7.4), 5% DMF cosolvent, at 20° C. Antibody complexes were made using 20 μM of mAb and 10 μM of 2. The mAbs were arranged in the order originally established by visual observation (Fig. 12). †The values in parentheses were observed as shoulders/inflections. ‡Quinine bisulfate in 0.5 M H₂SO₄ was used as a quantum yield reference with φ_{f} = 0.546 [J. N. | | | | | |
| # Demas, G. A. Crosby,J. Phys. Chem. 75,991 (1971)]. Flourescence emission spectra were collected for all complexes, free stilbenes and quinine sulfate from at least two different excitation wavelengths (313 nm and 327 nm). The quantum yields were accurate to ± 10%. §nd = not determined. ∥A second band was observed at 381 nm. In 2-methylcyclohexane, two bands were observed (356 nm and 376 nm). | | | | | |

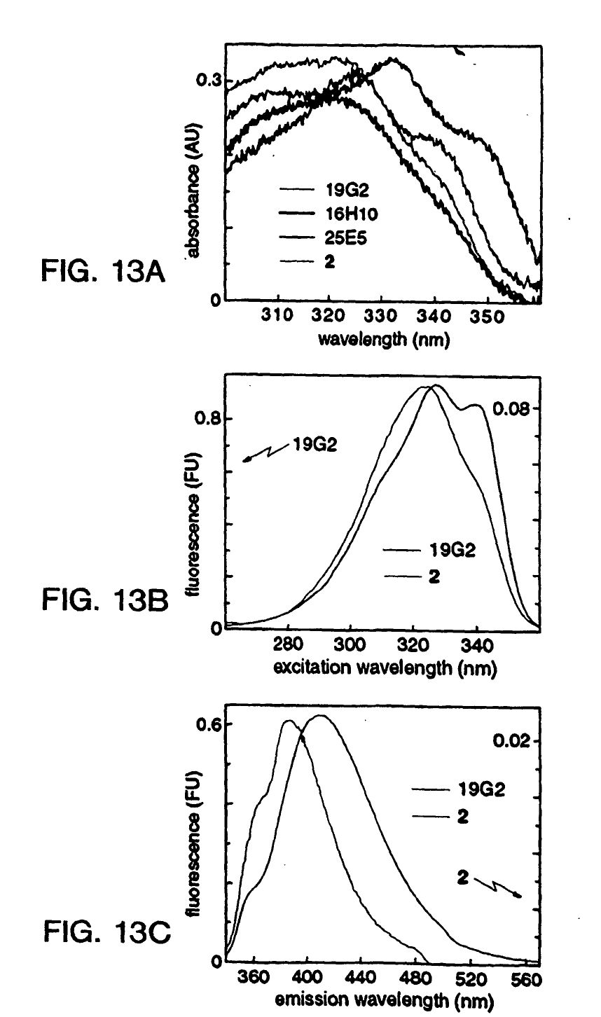

The room temperature absorption spectrum of the 19G2-2 complex was slightly red-shifted compared to free 2 and showed a vibronic progression of 0-0 and 0-1 sub-bands and a 0-2 sub-band as an inflection (FIG. 13A). The spectrum differed from 2 which showed very small inflections, that of 25E5-2 which lacked the 0-2 sub-band, and that of 16H10-2 which was featureless. The identification of vibronic bands was somewhat similar to, but much less defined than, 1 in viscous or low-temperature glassy media and stiff-stilbenes 9 and 10 at room temperature (J. Saltiel, J. T. D'Agostino, J. Am. Chem. Soc. 94, 6445 (1972); K. Ogawa, H. Suzuki, M. Futakami, J. Chem. Soc. Perk. Trans. II 39 (1988). These workers measured the spectra for 1, 9, and 10 in typically used low-temperature spectroscopic solvents 2-methylpentane and 2-methyltetrahydrofuran. In these organic solvents at room temperature, 1 showed a weak progression slightly more defined than 2 in the buffer solution. The band shapes for 1 in rigid media were much sharper and more defined than the 19G2-2 complex, but the vibronic progression was the same. The vibronic structure for 9 at room temperature was also better defined than 19G2-2, and with the 0-0 band intensity equal to 0-1. For 10, there was some additional fine structure in addition to the major bands. For 10 in buffer (Table 2), the fine structure was absent, but the progression more defined. In our low-temperature experiments, the excitation spectrum of 2 in frozen buffer at 100° K was more defined than the 19G2-2 complex. These workers observed that a slight red shift of the absorption spectrum of 1 occurred in more polar solvents and also for 9 and 10 compared to 1 in a particular solvent. We presumed that EP2 antibody binding sites would have a bulk dielectric constant less than that of aqueous buffer. However, localized effects were considered. Consequently, either structural phenomena or polarity effects, or both, could be invoked as the cause for the red shift in blue and blue-purple mAb complexes. The data suggested that 2 bound to 19G2 had a unique interaction with the antibody in the ground state, and was more planar and had less phenyl torsion in the ground and/or Franck-Condon excited states than free 2 or the blue-purple or purple mAb complexes. The excitation spectrum of the 19G2-2 complex showed structure analogous to the aborption spectrum (FIG. 13B). Emission of blue-fluorescent antibodies was broad and featureless, similar to the bandshape of 2 and other EP2 complexes, but with a red-shifted maximum at 410 nm that gave rise to the color that characterized these four mAbs (FIG. 13C). The strong emission and its spectral location was in stark contrast to free 2 which in fluid solution emitted in the near-UV with a φ_{f} that was 30-40-fold lower (Table 2). The change in overall appearance of the emission spectrum relative to the structured emission typically observed for trans-stilbene 1 as well as 2 in low-temperature rigid media (vide infra) suggested that the antibody caused a perturbation of the electronic structure of 2 and that the emission was due to a complex in the excited state. The eleven other EP2 mAbs gave rise to a smaller spectral shift (blue-purple and purple emission) and a lower φ_{f} of the overall emission. This further underscored the notable behavior of 2 bound to a blue-fluorescent antibody.

Somewhat unexpectedly, the panel of EP2 mAbs were able to bind the cis-isomer 6 (Table 1). (The compound 6 was prepared as follows. A solution of 4-nitrostilbene (trans/cis=1/8.5) (16) (774 mg, 3.43 mmol) in ethanol (12 ml) was reduced with SnCl₂—H₂O (3.88 g, 17.2 mmol) at room temperature for 3 h. The mixture was poured into EtOAc (200 ml), washed with water, saturated sodium bicarbonate, dried over sodium sulfate, filtered and evaporated. The residue was purified using flash chromatography (hexane/EtOAc=70/30) that afforded the product as a pale yellow oil (636 mg, 95%, trans/cis=1/2). Then, the cis isomer was purified before use using preparative thin-layer chromatography in the dark hexane/ether=50/50, developed twice). Glutaric anhydride (28.5 mg, 0.25 mmol) was added to a solution of cis-4-aminostilbene (39.4 mg, 0.20 mmol) in CH₂Cl₂ (2 ml) and stirred in the dark for 18 h. A solution of THF (2 ml) and CH₂Cl₂ (5 ml) was added and the mixture washed with water and brine. The organic layer was dried over sodium sulfate, filtered and evaporated that afforded 6 as a white solid (45 mg, 73%)). In general, the affinities were reduced with K_{d} values 10-fold higher. One interesting exception was mAb 9E 11 in which the K_{d} of the cis isomer was in fact slightly lower than that of the trans isomer. In retrospect, the cis-binding results were rationalized in light of the linker length of 2 used for immunoconjugate formation and immunization. Based on empirical data from our laboratory, linker lengths of 13-15 Å promoted complete, high affinity recognition of a variety of haptenic structures. Here, the linker length of 2 was approximately 8 Å between attachment to the KLH carrier protein and the proximal point of attachment on the stilbene framework. Consequently, while the distal aromatic ring and the connecting double bond were probably buried in the antibody binding site and served as the primary specificity determinants, there would be less recognition of the proximal aromatic ring depending on the particular mAb. Subsequent structural data (vide infra) supported the hypothesis. The model suggested a discrimination between the two rings in 2 or 6, perhaps different ground-state binding modes between cis and trans isomers, and that the proximal ring was “looser” and more subject to torsional effects. In light of the results, the possibility for interconversion of the two isomers at the antibody combining site was investigated.

First, the ground-state thermal effect of EP2 mAbs on 2 at 45° C. was examined. No evidence was found for the formation of 6 or any other new compound by either an exemplary blue-fluorescent mAb 19G2 or a purple-fluorescent mAb 16H10. This result was not surprising given the fact that the antibodies were elicited using a ground-state structure that contained no information about the transition state for catalysis of stilbene isomerization. Moreover, the ground-state barrier between trans and cis isomers of stilbene was found to be rather large at ˜43 kcal/mol (G. B. Kistiakowsky and W. R. Smith, J. Am. Chem. Soc. 56, 638 (1934)) that precluded spontaneous isomerization of 2 at the maximum 45° C. operating temperature that maintained antibody binding. While we could not rule out energy perturbations of the barrier or twist-angles on the ground-state surface, such effects were likely to be small and only minor contributors to the phenomenon of blue fluorescence.

Second, the photostationary state (pss) of 2 and 6 in the absence and presence of EP2 mAbs was measured (Table 1). The photoisomerization of either the traits or cis isomer alone in buffer solution at room temperature using the transilluminator (FIG. 12) produced an excess of cis-isomer 6 similar to the behavior of 1 and 5 in most solvents for excitation wavelengths >300 nm (H. Görner and H. J. Kuhn, Adv. Photochem. 19, 1 (1995). The photostationary state composition for direct excitation of a two-isomer system under a given set of conditions is a function of the product of extinction coefficients at a particular wavelength and the isomerization quantum yields: (trans/cis)ₚₛₛ=[ε_{c}(λ)/εₜ(λ)](φ_{cπt}/φ_{tπc}). UV irradiation of the cis-isomer 6 in the presence of EP2 mAbs afforded the same visual fluorescence as from 2. Spectroscopic analysis of free 6 and antibody complexes indicated nothing unusual and only an initial fluorescence from a small amount of the 2 present as an impurity. Fluorescence of 5 in fluid solution was only recently detected under special conditions. No isomerization of 2 was observed in routine spectroscopic samples as measured by HPLC (Table 1). Yet, in all cases, and especially blue-fluorescent mAbs, the final pss at the antibody active site favored the trans-isomer 2. Previous studies by others showed that in addition to 1 and 5 starting from either isomer, a minor photoproduct, dihydrophenanthrene, could form from the excited cis stilbene via electrocyclization (W. H. Laarhoven, in Photochonism: molecules and systems, H. Dürr, H. Bouas-Laurent, Eds. (Elsevier, N.Y., 1990), pp. 282-300). Although the latter process was reversible, the dihydrophenanthrene readily oxidized to phenanthrene 7 when oxygen was not excluded. Indeed, we detected the formation of 8 under normal conditions of sample preparation and irradiation in the presence of room air and surmised that this reaction manifold contributed to the bleaching of the blue fluorescence (vide supra). The slower rate of formation of 8 in both the background (no mAb) and EP2 mAb reactions compared to the rate of establishment of the pss allowed a “stable” pss to be attained before significant formation of 8. With blue-fluorescent mAbs at room temperature, 0 to <1% 8 was detected after 30-40 seconds starting with 2 and <1-1% at 30 seconds starting with 6. The results were similar for the other mAbs that had (trans/cis)ₚₛₛ>90/10. For some mAbs with higher levels of 6 in the pss, 8 was detected at 25 seconds and was generally 1-2% at 30 seconds starting from 2 or 6. In background reactions, <1-1% 8 was observed at 50 seconds starting with 2 and <1-1% at 40 seconds starting with 6. It was proposed that the initial motion of excited 5 on the S_{I} surface was towards photocyclization [H. Petek, et al., J. Phys. Chem. 94, 7539 (1990)]. The compound 8 had a K_{d}>500 μM and so did not compete with 2 or 6 at the pss concentrations (Table 1). However, it was uncertain whether oxidation of the corresponding dihydrophenanthrene occurred at the active site or in bulk solution. The formation of 8 was one possible pathway for photobleaching. Although under the conditions of the intensity of the UV illuminator, degradation of 8 was evident at 60 seconds and perhaps continued almost comparably to its rate of formation. At 60 seconds, mass balance began to deteriorate among 2, 6, and 8 concomitant with the loss of fluorescence. All compounds likely underwent oxidative and nonoxidative photoreactions. By chromatographic analysis, we observed no photoproduct formation, such as stilbene photodimers, but have preliminary evidence for covalent labeling of the antibody. Notably, a sample of 19G2-2 that was subjected to freeze-thaw evacuation cycles with argon purging and sealed in an ampoule was extremely photostable and completely bleached after seven hours of continuous UV irradiation. Only 8 remained, indicative that some oxygen had not been removed, at ˜15% of the initial concentration of 2. The compound 8 also served as a marker that suggested a small thermal barrier on the cis side of the pss. (Studies indicated that the isomerization of 1 had a ˜3.0 kcal/mol intrinsic barrier and was strongly influenced by solvent viscosity and to a smaller extent by temperature (J. Saltiel and Y.-P. Sun, in Photochronism: molecules and systems, H. Dürr, H. Bouas-Laurent, Eds. (Elsevier, N.Y., 1990), pp. 64-162; D. H. Waldeck, Chem. Rev. 91, 415 (1991); H. Görner and H. J. Kuhn, Adv. Photochem. 19, 1 (1995); J. Saltiel, J. T. D'Agostino, J. Am. Chem. Soc. 94, 6445 (1972)). From the cis side, isomerization has been assumed to be essentially barrierless subject only to medium-imposed factors (J. Saltiel and Y.-P. Sun, in Photochromism: molecules and systems, H. Dürr, H. Bouas-Laurent, Eds. (Elsevier, N.Y., 1990), pp. 64-162; D. H. Waldeck, Chem. Rev. 91, 415 (1991); H. Görner and H. J. Kuhn, Adv. Photochem. 19, 1 (1995)). The observation of small temperature effects in pss experiments suggested thermal barriers on both sides of the isomerization manifold for EP2 mAbs. At 45° C., the background pss showed no change from the value at 21° C., but the pss in antibody complexes shifted ˜2% in favor of cis-isomer 6. At −5° C., the 19G2-2 pss was >99% trans-isomer 2, and starting from 6 the first traces of phenanthrene 8 were observed before the establishment of the pss. Hence, formation of 8 became competitive with cis isomerization and resulted in a pss of 98% trans-isomer 2. Hence, the pss was actually a metastable equilibrium continuously shifted by the electrocyclization and irreversible dihydrophenanthrene oxidation, as well as other photodegradative pathways.

Both geometric restrictions and the effective polarity of a binding site can influence the relaxation pathways of a molecule in an excited state and alter the outcome of a reaction. Inclusion of trans-stilbene in β-cyclodextrin was previously examined by steady-state methods and found to favor a trans pss, yet interestingly showed no enhancement of fluorescence (M. S. Syamala, S. Devanathan, V. Ramamurthy, J. Photochem. 34, 219 (1986). The (trans/cis)ₚₛₛ=75/25. These workers proposed that interactions between 1 and the rim of the cyclodextrin occurred at the twisted ¹p* state. The cis-isomer 5 also isomerized and was postulated to bind in a different mode. However, an antibody binding site programmed by hapten design should be much more specific, dynamic, and chemically complex than a cyclodextrin cavity. Although a “lock and key” paradigm that invoked “freezing out” motions of 2 at the active site could in principle explain the increased φ_{f} values relative to 2 in buffer solution, the data did not support such a model. The absence of well-defined vibronic structure of absorption and emission bands were not indicative of a stilbene molecule with a rigidity able to furnish the φ_{f}˜0.7-0.8 of blue-fluorescent complexes. In this regard, the available binding energy of ˜8-9 kcal/mol should be sufficient to restrict the single-bond phenyl torsions (<<0.1 kcal/mol), but not the isomerization motion at the high energy of the excited state. Moreover, the effective viscosity at the active site of the antibody certainly should be much less than that of a frozen solvent or a medium such as glycerol (η=934 cp, 25° C.) that is 1000 times more viscous than water or hydrocarbons. Yet, remarkably, the φ_{f} for the 19G2-2 complex compared favorably with the value of φ_{f}=0.75 for 1 at 77° K in hydrophobic solvents and at 193° K in glycerol. In essence, the structural, chemical, and dynamic characteristics of the active-site matrix recapitulated a high friction or low-temperature “glassy” environment able to drastically reduce the isomerization funnel and efficiently yield fluorescence. Even blue-purple and purple complexes maintained high trans-favored pss and of values far greater than that of stilbene in fluid solution (Tables 1, 2). However, for neither blue, blue-purple, nor purple mAb complexes was the vibronic structure of spectra indicative of a rigid stilbene. While it was apparent that conformational mobility existed in all of these antibody complexes, the emission wavelengths were distinct. Accordingly, a picture emerged of unique, dynamically “tuned” interactions between stilbene and antibody bound together by noncovalent forces during the photoevent.

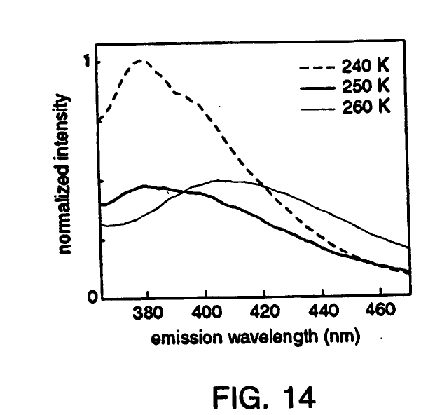

Vital support for the dynamic dependence of the blue fluorescence was obtained from low-temperature fluorescence spectroscopy that corroborated initial visual observations (vide supra). At 100° K, a structured emission of the 19G2-2 complex was observed comparable to 2 alone and reminiscent of trans-stilbene in rigid media. Temperature-dependent emission spectra were collected using a Janis Model SVT Research Cryostat mounted inside a Spex Fluoromax photon-counting fluorimeter using λₑₓ=355 nm for all spectra. Emissive lifetimes were collected with a Spectra-Physics Model GCR-150-10 Nd:YAG laser system. Third harmonic pulses were used for excitation λₑₓ=355 nm, energy<1 mJ, nominal pulse width=7 ns). [For details: N. H. Damrauer, T. R. Boussie, M. Devenney, J. K. McCusker, J. Am. Chem. Soc. 119, 8253 (1997); N. H. Damrauer and J. K. McCusker, Inorg. Chem. 38, 4268 (1999)]. The essential vibronic features of the emission profile remained intact through 220° K. The onset of a thermal transition began at 240° K where the intensity of the emission and vibronic pattern began to change dramatically (FIG. 14). Over the next 20° K, the spectrum broadened considerably and shifted to the red and at 260° K the evolution was complete and matched that observed at room temperature for the emission of blue-fluorescent antibodies. The abruptness of the transition with respect to temperature was notable especially since the bulk medium was still frozen at 260° K. Clearly, a dynamic event occurred near 250° K that allowed the conversion to the blue emissive species. In all likelihood, structural motion and/or vibrational states of the hapten were allowed in the region of 250° K due to a glass transition (D. Vitkup, D. Ringel, G. A. Petsko, M. Karplus, Nat. Struct. Biol. 7, 34 (2000)) of the antibody or specific coupled interactions between protein and hapten were activated in this temperature regime.

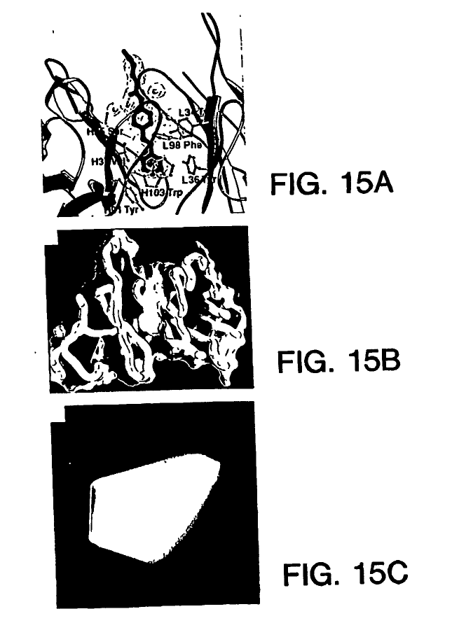

X-ray crystallography. Insight was obtained from structural analyses above and below the transition temperature. The structure of the Fab fragment of 19G2 complexed with 2 was solved to 2.4 A resolution at 4° C. (277° K) (FIGS. 15A, 15B). The Fab fragment of 19G2 was prepared by proteolytic digestion of the whole immunoglobulin followed by affinity purification. The Fab was treated with a three-fold molar excess of 2 in 10 mM Tris, 150 mM NaCl, pH 7.5, with 5% DMF cosolvent and crystallized by the hanging drop method from 100 mM sodium citrate, 300 mM MgCl₂, 12% PEG4000, 1 mM methionine, pH 4.5. A native data set was collected at SSRL, beamline 9-2, at 4° C. Data was processed with DENZO and SCALEPACK [Z. Otwinowski and W. Minor, Methods Enzymol. 276, 307 (1997)]. The structure was determined by molecular replacement techniques. The AMoRe package [J. Navaza, Acta Crystallogr. A50, 157 (1994)] was used to search a library of variable domains using data between 12.0 and 4.0 Å. The best solution for the variable domain was from pdb entry 1GGB. This model was used with the CCP4.40 program MOLREP [A. Vagin and A. Teplyakov, J. Appl. Cryst. 30, 1022 (1997)] to find the first variable domain (density/sigma 7.82) and then the second, NCS-related variable domain (density/sigma 16.7). Mutation of the molecular replacement model and rebuilding was done using the program 0 [T. A. Jones, J.-Y. Zou, S. W. Cowan, M. Kjeldgaard, Acta Crystallogr. A47, 110 (1991)]. Refinement was carried out using positional, simulated annealing and torsional refinement in CNS [A. Brunger, et al., Acta Crystallogr. D54, 905 (1998)] with NCS restraints turned on and bulk solvent corrections applied between 20.0 and 6.0 Å. Interim statistics: space group, C2; unit cell dimensions, a=196.9 Å, b=62.1 Å, c=93.5 Å and α=γ=90°, β=117.5°; resolution range, 20.0-2.42 Å; observations, 36035; completeness, 94.7%; I/σ, 29; Rmerge, 3.8%; final resolution bin 2.51-2.42 Å, completeness, 70%, Rmerge, 29.5%, I/σ, 5.4; refined residues, 854; refined water molecules, 204; Rcryst, 0.238%; Rfree, 0.263%; bond length deviation, 0.0072 Å; bond angle deviation, 1.59°). Interestingly, as for the 19G2-2 complex in solution at this temperature, the crystals glowed blue upon UV irradiation (FIG. 15C). The hapten was readily modeled into the density in a planar, trans-configuration with the long axis directed towards the center of the antibody and all of the nonhydrogen atoms accounted for in an Fₒ-F_{c} difference map. Most of the side-chains that packed against the stilbene moiety were nonpolar in nature. The phenyl ring distal from the linker was positioned primarily by a “face-to-face” π-stacking interaction with the indole group of the heavy-chain tryptophan 103 (Kabat numbering), a residue generally invariant in the amino acid sequence of all antibodies. Indeed, W103 was also present in the sequence of the purple antibody 16H10. The central olefinic carbons of the stilbene were enclosed by heavy-chain residues V37 and A93 and light-chain residues Y36 and F98 in which the hydroxyl group of Y36 was within 3.3 Å of the distal carbon. The proximal phenyl ring was positioned between the loops of complementarity-determining regions H3 and L3 with heavy-chain G95 and light-chain P96 on either side of the ring. Notably, the protein packing in the region of the proximal ring was less intimate compared to that of the distal ring. Three water molecules were anchored by main-chain and side-chain hydrogen bonds to form part of the van der Waals surface against the proximal ring. Finally, the crystallographic structure of the 19G2-2 complex at low temperature (100° K) indicated that the positions of the stilbene and of all main-chain and side-chain atoms of the antibody active site were identical to that of the complex at 4° C., above the 250° K transition temperature. Consequently, while it now seemed reasonable to invoke an exciplex involving W103, it was also evident that blue fluorescence from such an interaction must arise by unique dynamic interplay between the stilbene hapten and antibody in the excited state.

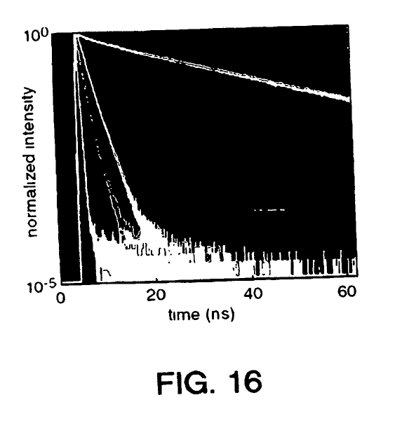

Dynamic spectroscopy. Picosecond time-resolved emission spectroscopy was used to further probe the dynamics of blue-fluorescent antibodies. Time-resolved emission decay profiles of free 2 and bound to EP2 mAbs were measured at room temperature by time-correlated single-photon counting (Table 3, FIG. 16).

TABLE 3
Time-resolved data for mAb complexes and stilbenes.*

| EP2 | fluorescence decay | τᵣ | kᵣ | kₙᵣ |
|---|---|---|---|---|
| mAb | components, τ_{f} (ns)† | (ns)‡ | (ns⁻¹)§ | (ns⁻¹)∥ |
| 19G2 | 22.9, 7.6, 1.1, 0.086 | 31 | 0.032 | 0.0091 |
| 20F2 | 23.3, 7.5, 1.05, 0.092 | 31 | 0.032 | 0.0081 |
| 22B9 | 22.9, 7.2, 0.97, 0.07 | 36 | 0.028 | 0.012 |
| 21C6 | 23.2, 9.1, 1.5, 0.27 | 39 | 0.026 | 0.014 |
| 25F8 | 1.48, 0.926 | 3.9 | 0.26 | 0.16 |
| 25E5 | 1.66, 1.06 | 2.6 | 0.38 | 0.23 |
| 23E4 | 1.92, 1.09 | 4.9 | 0.20 | 0.17 |
| 23G3 | 1.85, 1.06 | 4.9 | 0.20 | 0.15 |
| 23D3 | 0.915, 0.397 | 4.3 | 0.23 | 0.52 |
| 23C2 | 0.937, 0.408 | 5.2 | 0.19 | 0.58 |
| 25C10 | 0.904, 0.384 | 4.9 | 0.20 | 0.55 |
| 24B6 | 1.38, 0.571 | 4.1 | 0.24 | 0.29 |
| 21E2 | 1.71, 0.885 | 4.8 | 0.21 | 0.16 |
| 16H10 | 0.864, 0.375 | 4.3 | 0.23 | 0.77 |
| 9E11 | 0.720, 0.154 | 4.5 | 0.22 | 1.1 |
| 2 | 0.071 | 3.6 | 0.28 | 14 |
| 10 | 1.71¶ | 3.5 | 0.29 | 0.30 |
| *Unless otherwise noted, all measurements were made in PBS (10 mM sodium phosphate, 150 mM NaCl, pH 7.4), 5% DMF cosolvent, at 20° C. Antibody complexes were made using 20 μM of mAb and 10 μM of 2. The mAbs were arranged in the order originally established by visual observation (FIG. 12). †Time-resolved fluorescence decay profiles were recorded using the picosecond dye laser and time-correlated single photon counting system described | | | | |
| # elsewhere [C. R. Guest, R. A. Hochstrasser, L. C. Sowers, D. P. Millar, Biochemistry 30, 3271 (1991)]. Decay curves were fitted as a sum of exponential decays: I(t) = Σᵢ αᵢ exp(−t/τᵢ), where αᵢ and τᵢ are the decay amplitude and decay time of component i. For fitting of experimental decays, the summation on the right hand side of this equation was convoluted with the instrumental response | | | | |
| # function. The values of αᵢ and τᵢ were adjusted for best fit. The best fits were obtained using the fluorescence decay times shown as judged by the value of x² and by examination of weighted residuals. The component making the dominant contribution to the total emission had the longest lifetime except for mAb 25E5 in which the shorter-lived component was dominant. ‡The radiative lifetime (τᵣ) was calculated | | | | |
| # from the fluorescence lifetime (τ_{f}) and quantum yield (φ_{f}) (Table 2): τᵣ = τ_{f}/φ_{f}. In the case of multiexponential fluorescence decays as observed for the EP2 complexes, a good approximation for τᵣ was obtained if the decay component τᵢ with the largest relative weight φ_{f} (rel, i) was used in a slightly modified equation: | | | | |
| # τᵣ = τᵢ/[φ_{f} φ_{f} (rel, i)]. The decay component with the largest relative weight at a given wavelength was calculated using the equation: φ_{f}(rel, i) = (αᵢ τᵢ)/(Σᵢ αᵢ τᵢ) [W. Rettig, W. Majenz, R. Herter, J. F. Letard, R. Lapouyade, Pure Applied Chem. 65, 1699 (1993)]. §Radiative rate constant. Calculated as the inverse of τᵣ. | | | | |
| # ∥Nonradiative rate constant. Calculated as follows: kₙᵣ = [kr(1-φ_{f})/φ_{f}]. ¶Measured in 2-methylcyclohexane. | | | | |

The hapten 2 in aqueous buffer exhibited a rapid decay with one fluorescence lifetime of ˜70 ps, in good agreement with previous data for 1 under comparable conditions, that indicated similar excited-state decay pathways for the two molecules. A dramatic change in the excited-state lifetime was observed for complexes of 2 with blue-fluorescent mAbs. In striking contrast to the sub-nanosecond lifetime of 2 in solution, the decay profile of the complex was dominated by an unusually long lifetime of 23 ns (Table 3). However, the blue-purple (e.g., 25E5) and purple (e.g., 16H10) complexes exhibited maximum fluorescence decay times that did not exceed 2.0 ns (Table 3). In all complexes, the existence of multiple decay times probably represented heterogeneity in the ground and/or excited states. Other workers observed two decay times for 1 complexed with β-cyclodextrin and regarded these as average fluorescence lifetimes of loosely bound and tightly bound forms that interconverted slowly on the time scale of the photoisomerizafion [G. L. Duveneck, E. V. Sitzmann, K. B. Bisenthal, N. J. Turro, J Phys. Chem. 93, 7166 (1989)].

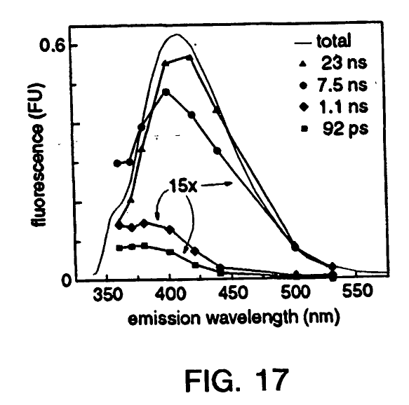

Significantly, decay-associated spectra of a blue-antibody complex revealed pronounced spectral differences among the four lifetime components (FIG. 17). The spectra corresponding to the two shortest lifetimes were centered around 380 mm, coincident with the emission spectrum of free 2 or the low-temperature (240° K) emission spectrum of the complex (FIG. 14), whereas the spectra corresponding to the two longest lifetimes were red shifted to around 420 nm. Based on the red-shift and the long decay times the 420 nm emission was interpreted as an exciplex of 2 with an antibody residue that was likely W103, while the 380 nm emission was assigned to stilbene 2 itself at the antibody combining site.

Exciplex emission was further supported by the calculation of radiative lifetimes (Σᵣ) for blue-antibody complexes. The Σᵣ for 2 in aqueous buffer was 3.6 ns (Table 3) consistent with previous determinations of the radiative lifetime of trans-stilbene 1 (J. Saltiel, A. S. Waller, D. F. Sears, Jr., C. Z. Garrett, J. Phys. Chem. 97, 2516 (1993); J. B. Birks, D. J. S. Birch, Chem. Phys. Lett. 31, 608 (1975)). However, the τᵣ for the blue-fluorescent mAbs was 30-40 ns or one order of magnitude larger than the intrinsic value. In contrast, the τᵣ values for the blue-purple and purple complexes were comparable to free 2 and not that expected of an exciplex. Long radiative lifetimes (>3.6 ns) have been observed for exciplexes and excimers of 1 in solution. Excimer fluorescence of 1 as a ternary complex with solvent in γ-cyclodextrin [R. A. Agbaria, E. Roberts, I. M. Warner, J Phys. Chem. 99, 10056 (1995)] and in hybrid oligonucleotides (F. D. Lewis, T. Wu, E. L. Burch, D. M. Bassani, J.-S. Yang, S. Schneider, W. Jägr, R. L. Letsinger, J. Am. Chem. Soc. 117, 8785 (1995)] was reported. However, in a specific binding site generated by hapten programming, complexation of multiple stilbenes 2 would be precluded. Exciplex formation between stilbenes and amines has been thoroughly investigated. [For example: F. D. Lewis et al., J Am. Chem. Soc. 117, 660 (1995)). Since primary amines did not participate, this tended to rule out the possible involvement of a lysine residue. Furthermore, the absence of pH effects on 19G2-2 fluorescence also argued against dependence on a particular ionic state of the protein (i.e., charged residues). However, we could not exclude the possibility of exciplex formation with aromatic amino acids. Finally, we did not detect the existence of a 2 radical or cation-radical species using electron-spin resonance (ESR) under steady-state irradiation [For 1 see: J. L. Courtneidge, A. G. Davies, P. S. Gregory, J. Chem. Soc. Perkin Trans. 2 1527 (1987)]. Significantly, the radiative lifetime of a stilbene exciplex must be longer than that of stilbene alone because of a low quantum-mechanical probability of the transition from the exciplex state to the ground state.

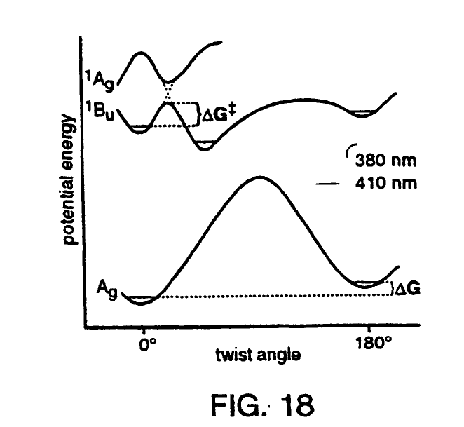

Finally, the kinetics of exciplex formation was directly monitored in a time-dependent fashion. An examination of the temporal behavior at the long- and short-wavelength extremes of the spectrum showed that the appearance of the blue exciplex emission was coupled to the initial decay of the purple stilbene emission (FIG. 18). Hence, excitation of 2 resulted in a short-lived stilbene fluorescence followed by evolution to a blue-emitting exciplex that persisted for tens of nanoseconds and produced more than 98% of the observed emission (Table 3).

Nature of the exciplex. Based on the results presented herein, we suggest that the emission of blue-fluorescent antibodies was due to the formation of an exciplex dynamically established between the stilbene 2 and an active-site residue during the photoevent. The most likely protein partner appeared to be W103-positioned in a π-stacking orientation with respect to the distal aromatic ring of 2. In addition, the hydrophobic environment of the antibody active site was conducive to a charge-transfer exciplex that was stable, long-lived, and afforded a high quantum yield. (Solvent effects on indole-aromatic exciplexes were reported [J. P. Palmans, M. Van der Auweraer, A. M. Swinnen, F. C. De Schryver, J. Am. Chem. Soc. 106, 7721 (1984)]. However, as revealed by the temperature dependence and crystal structure data, the critical aspect was that a simple static interaction between stilbene 2 and tryptophan was not sufficient to yield the blue-fluorescence.

At low temperatures (<250° K), the hapten bound to 19G2 was completely restricted with regard to transπcis isomerization. The resulting emission therefore resembled that typically observed for trans-stilbene in a rigid matrix such as an optical glass. As the temperature was increased, a transition occurred at 250° K that allowed formation of the exciplex and emission of blue light. Notably, the x-ray structural data showed no change in the protein structure nor any significant repositioning of antibody residues or stilbene hapten in the binding site above or below the transition temperature. Therefore, stilbene and antibody must be dynamically coupled to yield the exciplex. In fact, it was possible to follow this event in real time which took place in ˜80 ps at room temperature. Consistent with this model was the observation of slight differences (φ_{f} and τᵣ) in the emission of the four blue antibodies, since these parameters might be sensitive to the relative positions of hapten and partner residue(s) that will vary somewhat amongst mAbs. In this regard, we also cannot rule out that the exciplex resulted from a higher-order ensemble of stilbene and multiple aromatic residues that were evident in the crystal structure. While ternary or higher exciplexes would be entropically disfavored in solution, the union of hapten within the ordered protein scaffold would make tenable a scenario involving a π-network. Noteworthy, although W103 was present in the blue antibodies, it was also present in the purple mAb 16H10. Although the x-ray data for 16H10 remains to be acquired, the position of W103 should not be grossly different based on the consistency of antibody structure. Hence, subtle structural variations and mechanistic details were perhaps most important in dictating the photophysics and photochemistry of antibody-stilbene complexes.

A physicochemical mechanism for the blue fluorescence would imply that following photoexcitation above 250° K the bound substrate 2 underwent partial twisting along the trans/cis isomerization coordinate and at some point interacted with the appropriately positioned ryptophan residue. Below 250° K, despite no apparent static differences in the relative positions of 2 and W103, the motion required for creation of the exciplex was hindered and so blue fluorescence was not observed. One possible reason for efficient dynamic coupling of the two aromatic moieties would entail favorable changes in electron demand or redox potential that occurred as the stilbene molecule reorganized its electronic state. Alternatively, specific vibrational modes of the protein might mediate coupling between the exciplex partners. It is tempting to speculate that a unique set of protein dynamics, selected from the immune repertoire, facilitated the mixing of molecular orbitals through the interaction of resonant vibrational modes. Regardless, the observed phenomenon was intrinsic to the protein and stilbene in that no design elements were implemented to control the excited state. Yet, the antibody was able to dynamically accommodate and productively funnel a large amount of energy into a long-lived fluorescent species with little thermal loss into the protein matrix.

Proteins and catalysis. Although most biological processes occur on the thermally controlled ground-state surface, photochemical reactions involving molecular excited states are fundamental in Nature. In fact, all life is founded on the photosynthetic machinery of plants and some bacteria that contain large chromophores embedded in protein structures. Similarly, the physics and chemistry of vision is mediated by the light-induced double-bond isomerization of 11-cis-retinal bound to the protein rhodopsin (G. G. Kochendoerfer, S. W. Lin, T. P. Sakinar, R. A. Mathies, Trends Biochem. Sci. 24, 300 (1999)). The application of modern spectroscopic techniques to these photon-driven reactions, as well as other nonphotochemical systems, has allowed the observation of ultrafast and efficient mechanisms and shed light on a crucial paradigm for catalysis. Namely, protein-ligand interactions are dynamic and intimately dependent on the transfer of vibrational energy.

Blue-fluorescent antibodies provided both visual and spectroscopic evidence for quantum dynamic effects in protein reaction chemistry. Hence, antibodies are able to control energetic manifolds not only in the ground state, but also dramatically influence excited-state surfaces. Molecules that are promoted to electronically excited states by the absorption of light have a number of reaction pathways that may be traversed. Binding energy, mediated by specific amino acid contacts, can serve as a linchpin between two dynamic entities, protein and ligand. Blue-fluorescent antibodies revealed the exquisite capacity for a finely-tuned thermochemical interaction to efficiently produce a quantum molecular event. In this regard, a first step was taken toward utilizing photochemical sensors to study the ways that proteins catalyze reactions particularly in terms of the role of quantum chemistry and dynamics. Practical applications should also be possible and include the use of blue-fluorescent antibodies for the detection of DNA gene sequences using nonnatural stilbene nucleobases.

Protein-ligand interactions are invoked in virtually all biological processes. So, in the broadest sense, biological catalysis encompasses any function otherwise insufficient or unattainable in the absence of a protein. Each protein is a unique environment that maintains a highly complex ensemble of possible relaxation pathways and of different vibrations within the surrounding thermal bath. There is growing appreciation for the need of a theory that is quantum mechanical and anharmonic to study the manner in which vibrational energy can drive physical and chemical reactions in proteins. Indeed, an often unaccounted for force in biological catalysis might reside in the resonance energy between protein and ligand available from coupled vibrations of the same intrinsic frequency.

### EXAMPLE 2

### Nucleosides Containing Stilbene

The synthesis of stilbene-tethered hydrophobic C-nucleosides is described. Compounds of this type are targeted for use with our recently reported “blue-fluorescent antibodies” with the aim of probing native and nonnatural DNA. The nucleophilic addition of aryl Grignard reagents to either a protected 2′-deoxy-1′-chloro-ribofuranose or a protected 2′-deoxy-ribonolactone was the key synthetic step and afforded C-nucleosides in good yields. Both routes resulted in a final product that was ≧90% of the β-anomer. Amide- and ether-based linkers for attachment of trans-stilbene to the nucleobase were assessed for utility during synthesis and in binding of the ligands to a blue-fluorescent monoclonal antibody. X-ray structures of each complex were obtained and serve as a guideline for second-generation stilbene-tethered C-nucleosides. The development of these hydrophobic nucleosides will be useful in current native and nonnatural DNA studies and invaluable for investigations regarding novel, nonnatural genomes in the future.

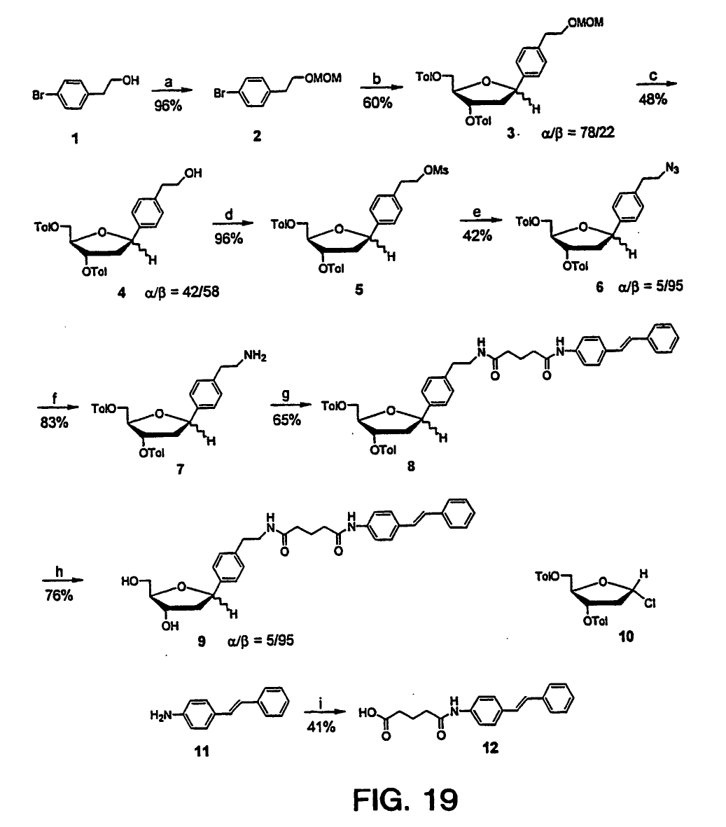

Two substituted benzene C-nucleosides were prepared that differed in the composition of the linker between stilbene and nucleobase. Each linker type could have unique advantages during polymer-supported oligonucleotide synthesis or in enzymatic reactions. In addition, two synthetic approaches were explored to assess their utility for future work. In the preparation of the first compound 9, the key step depended on formation of the Grignard reagent derived from 2 and its coupling with 1,2-dideoxy-3,5-di-O-p-toluoyl-α-1-chloro-D-ribofuranose 10 (FIG. 19).

These substitution reactions do not proceed with inversion, but yield a mixture of isomers (anomers) in which the α-isomer is predominant. Here, the nucleobase was installed to give 3 in 60% yield comprised of 78% of the a-anomeric configuration. Notably, the yield was better than generally observed for such couplings. Assignment of α- and β-isomers was carried out using ¹H NMR in conjunction with literature data. For aromatic C-nucleosides, α-isomers are characterized by an apparent triplet (J=6-7 Hz) and β-isomers by a doublet of doublets (J=10-11, 5-6 Hz) for proton 1′-H on the deoxyribose ring. Since the α-isomer is undesirable for our DNA studies, it was necessary to isolate the β-isomer. However, upon examination of 3, no separation was evident by thin-layer chromatography. To make the anomer ratio more favorable, the mixture was subjected to acid-catalyzed isomerization that resulted in a new mixture 4 now slightly enriched in the β-isomer. Finally, upon formation of the azido compound 6, one chromatographic operation could be used to separate the two closely eluting anomers to yield pure material that was 95% β-isomer. Reduction of the azido group with triphenylphosphine followed by EDC-mediated amide bond formation with 12, the original substrate for blue-fluorescent antibodies, afforded the protected compound 8. Hydrolysis of the p-toluate esters resulted in 9, the first of our stilbene-tethered C-nucleosides.

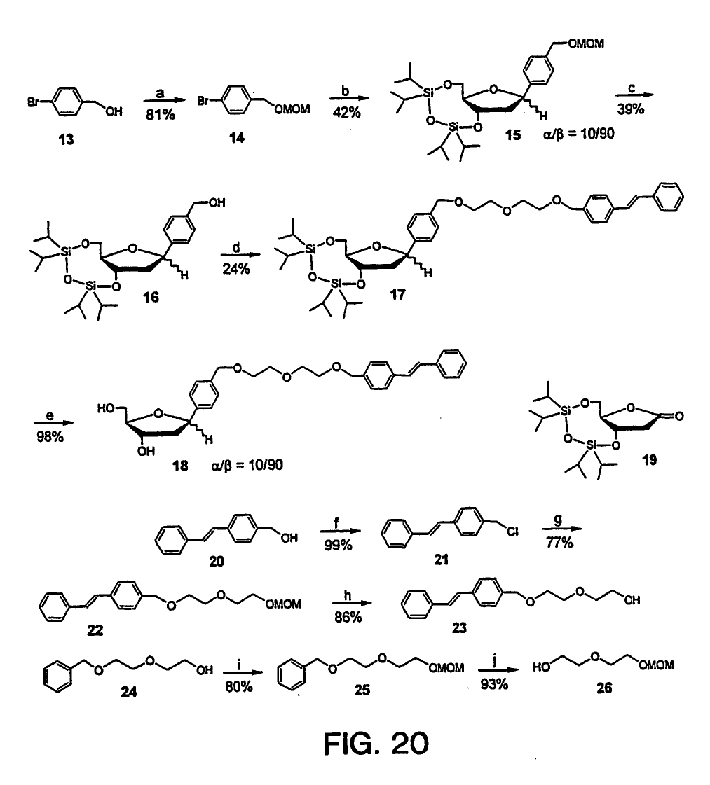

A second C-nucleoside incorporating an alternative linker was similarly founded on the most fundamental structure involving a benzene nucleobase. In this case, the para-substituted aromatic ring was introduced using recent methodology developed by Woski and coworkers that utilized the ribonolactone 19 (FIG. 20).

Unlike 10, the lactone is a very shelf-stable reagent suitable for long-term storage. Furthermore, organometallic additions to 19 result in an anomeric mixture with a high percentage of the P-configuration. The drawbacks of the approach are that the initial addition gives a hemiketal which requires a silane reduction operation and that the overall yield for the C-nucleoside is generally lower. After coupling to obtain 15, selective removal of the methoxymethyl ether with TMSBr afforded the deprotected alcohol. For this synthesis, we used a benzylic alcohol as a nucleobase so that a glycol linker could be attached via conversion of 16 to the trifluoromethane-sulphonate followed by reaction with 23. Although the yield was poor, no other methodology was successful for ether formation in these compounds. Fluoride cleavage of the tetraiso-propyldisiloxane of 17 afforded the C-nucleoside 18 that was 90% β-isomer. At no point in the synthesis could the anomers be distinguished by silica-gel chromatography, and so the final product ratio was fixed by the addition to 19 and hydride reduction of the hemiketal. Whether the inability to separate isomers will be a general occurrence using this route, remains to be determined. However, several cases were reported where the β-isomer was obtained as >90% of the mixture which bodes well for other C-nucleosides in our plans.

Each C-nucleoside was tested for the ability to bind to the blue-fluorescent monoclonal antibody (mAb) 19G2. Indeed, the bright, powder-blue fluorescence characteristic of the mAb 19G2-stilbene interaction was observed, and the quantum yield (φ_{f}) for each of the two complexes were comparable to that measured previously for 19G2-12 with a value φ_{f}˜0.80. Also, soaking a crystal of mAb 199G2 with 9 or 18 resulted in the blue emission. Subsequently, we acquired X-ray crystallographic data on both complexes primarily to obtain information regarding positioning of the nucleobase moiety with regard to the protein framework. The structures of the 19G2-9 and 19G2-18 complexes were determined to a resolution of 2.45 Å and 2.20 Å, respectively (Table 4). In both cases, the antibody structures differ by an RMSD of 0.61 Å and 0.50 Å from that of the previously determined 19G2-12 complex. The stilbene portion of both 9 and 18 is clear in 2σ density and could be readily modeled into the density as the trans-isomer. For both complexes, binding site amino acid residues within 5 Å of the stilbene are observed to be in the same conformation as in 19G2-12.

TABLE 4
X-ray data for antibody complexes.

| parameter | 19G2-9 | 19G2-18 |
|---|---|---|
| molecular | | |
| spacegroup | C2 | C2 |
| a (Å) | 196.352 | 194.651 |
| b (Å) | 60.613 | 60.840 |
| c (Å) | 93.039 | 92.498 |
| α(°) | 90 | 90 |
| β(°) | 117.5 | 117.3 |
| γ(°) | 90 | 90 |
| data collection | | |
| resolution (Å) | 20-2.45 | 20-2.2 |
| | (2.54-2.45) | (2.28-2.20) |
| observations | 33485 | 47669 |
| completeness (%) | 92.9 (90.8) | 96.7 (92.5) |
| I/σ | 23.1 (4.6) | 22.4 (2.8) |
| Rmerge (%) | 6.1 (26.4) | 7.0 (39.7) |
| refinement | | |
| Rwork | 0.260 | 0.265 |
| Rfree | 0.319 | 0.293 |
| bond lengths (Å) | 0.007 | 0.015 |
| bond angles (°) | 1.3 | 1.8 |

(RMSD for both bond lengths and bond angles)

In 19G2-9, the electron density is clear for 7.5 Å from the stilbene out to the second amide group of the linker and then diverges in multiple directions. This indicates that the linker terminus and appended C-nucleoside assume multiple conformations at the outer rim of the antibody binding site. The two conformations most obvious from the electron density were modeled and diverge about 60° away from one another, but both bring the nucleoside analog within close proximity of some parts of the complementarity determining regions (CDRs). One conformation interacts almost exclusively with the heavy-chain CDR3 (H-CDR3), and the other interacts with H-CDR3, as well as H-CDR1 and H-CDR2. Both conformations form hydrogen-bonding interactions between the glutaric-amide linker and the antibody, either to the side-chain amide nitrogen of the H96 Gln in one mode, or to the backbone carbonyls of light-chain 91 (L91) Asn and L92 Leu in the other conformation. The C-nucleoside fragment is also within hydrogen bonding distance of antibody functional groups, but it is likely that the mobility generates conformations in addition to what is modeled. Although electron density exists for the C-nucleoside in either conformation, the density is not sufficient to confidently place the deokyribose or phenyl groups into an exact orientation and the B-factors in this region are relatively high.

Similarly, in the 19G2-18 complex the linker and C-nucleoside region can be modeled in at least two different conformations placed almost 180° away from one another. One of the conformations assumes a position near H-CDR3 as observed in 19G2-9, whereas the other conformation interacts primarily with the L-CDR3 loop, which was not observed in 19G2-9. No hydrogen bonds are formed to the ligand prior to the divergence of the conformations. However, the first conformation forms a hydrogen bond between the last ether oxygen of the linker and the backbone oxygen of L92 Leu and both conformations bring the C-nucleoside within hydrogen-bonding distance of the antibody. The deoxyribose group can form a hydrogen bond to the guanidinium group of H94 Arg and the backbone oxygen of H96 Asn in one conformation, and in the other comes within hydrogen-bonding distance of the L92 Leu backbone oxygen. As was the case with 19G2-9, the high B-factors for the C-nucleoside are probably indicative of a wide range of possible conformations.

Our previous work on 19G2-12 suggested that the linker lengths employed in 9 and 18 would allow complete immersion of the stilbene moiety in the binding site, while retaining the C-nucleoside portion at the precipice. The X-ray structures show this to be the case. This now serves as a guideline for our second-generation designs in which the linker length will be increased in order to ensure the complete emergence of trans-stilbene from the ˜8.5 Å deep major groove of double-helical DNA enabling recognition by mAb 19G2. We anticipate that stilbene-tethered C-nucleosides will be compatible with duplex formation if the linker is of sufficient length and flexibility to avoid steric congestion of the appended stilbene with the DNA strands and/or the active site of a DNA-utilizing enzyme. In this way, an appropriate linker might allow for base pairing and nucleoside ligation during DNA synthesis.

In the work described, we have provided a proof-of-principle for construction of a new class of compounds of potential value in both current and future DNA and genomic studies. The structures as presented are readily amenable to activation as the 2′-OH phosphoramidite for oligonucleotide synthesis or formation of the 5′-OH triphosphate for use as DNA polymerase or reverse transcriptase substrates using well-established methods. Furthermore, it is possible to prepare the C-nucleosides with a ribose, rather than deoxyribose sugar, using a similar approach that would afford substrates for RNA polymerases. The exo-nuclease deficient Klenow fragment of E. coli DNA polymerase I is able to efficiently recognize a large number of nonnatural hydrophobic bases and incorporate them into DNA. In this way, mapping and chain-termination sequencing could be used in a fashion similar to current protocols. Hybridization, widely used in high-throughput genomics strategies, would also be feasible. Yet, a significant advance will come from polymerase-mediated extension of DNA containing the nonnatural base, at present a hurdle in most cases, for synthesis of read-through or runoff transcripts/reverse transcripts. Ultimately, with regard to both nonnatural DNA and genomes in the years to come, a sequencing methodology will be needed comparable to what is now routine with natural DNA. Finally, targeting DNA with a macromolecular marker has unique advantages associated with the ability to apply immobilization technology for fragment isolation and recovery. Continued developments in nonnatural nucleobase design and the protein engineering of polymerase substrate specificity and activity will eventually provide a unique set of tools for the investigation of genetic material.

General Methods. ¹H and ¹³C NMR spectra were measured on a Brucker AMX-400 or Brucker AMX-500 spectrometer as indicated. Chemical sifts (ppm) were reported relative to internal CDCl₃ (¹H, 7.26 ppm and ¹³C, 77.0 ppm) CD₃OD (¹H, 3.30 ppm and ¹³C, 49.2 ppm) and DMSO-d₆ (¹H, 2.49 ppm and ¹³C, 39.0 ppm). HRMS spectra were recorded using electrospray ionization (ES) or MALDI techniques. Glassware and solvents were dried by standard methods. Flash chromatography was performed on silica gel 60 (230-400 mesh) and thin-layer chromatography on glass plates coated with a 0.02 mm layer of silica gel 60 F-254. All chemical reagents and solvents were from Aldrich Chem. Co., unless otherwise noted, and used without further purification.

4-[2-(methoxymethoxy) ethyl] bromobenzene (2). To a solution of 4-bromophenethyl alcohol 1 (1.0 g, 5.0 mmol) in dimethoxymethane (10 ml), were added LiBr (87 mg, 1.0 mmol) andp-TsOH—H₂O (95 mg, 0.50 mmol) with stirring. The white suspension was stirred at room temperature for 2 h or until completion of the reaction (tlc; hexane/EtOAc, 4/1). Brine was added and the mixture was extracted with ether. After evaporation of the solvent, the crude product was purified using flash chromatography (FC) (hexane/EtOAc, 4/1) to afford 1.17 g (96%) of 2 as a colorless oil. ¹H NMR (CDCl₃, 500 MHz) δ 7.41 (d, 2H, J=8.0 Hz), 7.12 (d, 2H, J=8.0 Hz), 4.60 (s, 2H), 3.74 (t, 2H, J=7.0 Hz), 3.29 (s, 3H), 2.86 (t, 2H, J=7.0 Hz). ¹³C NMR (CDCl₃, 125 MHz) 138.0, 131.4, 130.6, 120.0, 96.4, 68.0, 55.2, 35.7.

1,4-anhydro-2-deoxy-1-C-[4-[2-(methoxymethoxy) ethyl] phenyl]-D-erythro-pentitol 3,5-bis (4-methylbenzoate) (3). A solution of 2 (0.967 g, 3.95 mmol) in THF (4 ml) was added into a flask charged with Mg powder and a few crystals of iodine at room temperature under nitrogen. The mixture was stirred at 50° C. for 2 h to complete the formation of the Grignard reagent. A solution of chlorosugar 10 (1.23 g, 3.16 mmol) in THF (8 ml) was added at 0° C. and the reaction mixture was stirred at room temperature for 12 h. The mixture was concentrated to a small volume. The residue was purified by FC (hexane/EtOAc, 4/1) to give 1.0 g (60%) of 3 as an oil that was a mixture of isomers (α/β=78/22). ¹H NMR (CDCl₃, 500 MHz) δ 7.93-7.71 (m, 4H), 7.36-7.32 (m, 2H), 7.28-7.17 (m, 6H), 5.61-5.58 (m, 1H, α-isomer and β-isomer 3′-H), 5.34 (t, 1H, α-isomer 1′-H, J=6.6 Hz), 5.23 (dd, 1H, β-isomer 1′-H, J=11.4, 5.5 Hz), 4.69-4.52 (m, 5H); 3.77-3.73 (m, 2H), 3.30 (s, 3H), 2.96-2.88 (m, 3H, benzylic, α-isomer 2′-Hβ), 2.51 (dd, 1H, β-isomer 2′-Hα, J=13.2, 4.4 Hz), 2.44-2.40 (m, 6H), 2.32-2.20 (m, 1H, α-isomer 2′-Hα, β-isomer 2′-Hβ). ¹³C NMR (CDCl₃, 125 MHz) δ 166.3, 166.2, 166.1, 166.0, 165.9, 144.0, 143.8, 143.7, 143.6, 140.1, 138.8, 138.5, 138.4, 138.0, 138.8, 130.0, 129.6, 129.5, 129.1, 129.0, 128.9, 128.8, 128.7, 127.0, 126.9, 126.8, 125.9, 125.7, 125.6, 96.3, 82.8, 81.9, 80.6, 80.0, 77.2, 76.3, 68.4, 68.2, 64.7, 64.5, 55.0, 41.6, 40.2, 35.8, 21.6, 21.5. MALDI-FTMS: calcd for M+Na⁺ 541.2197, found 541.2211.

1,4-anhydro-2-deoxy-1-C-[4-(2-hydroxyethyl) phenyl]-D-erythro-pentitol 3,5-bis (4-methylbenzoate) (4). Compound 3 (1.0 g, 1.9 mmol) was dissolved in MeOH (25 ml) with one drop of 37% HCl. The mixture was stirred at 65° C. After completion of the reaction in 6-8 h, the solvent was evaporated. The residue was purified by FC (hexane/EtOAc, 2:1) to afford 0.82 g (90%) of a colorless syrup (α/β=73/27). The compound (0.82 g, 1.7 mmol) was epimerized in toluene (50 ml) with benzenesulfonic acid (30 mg, 0.17 mmol), conc. H₂SO₄ (1 drop), and water (3 drops). The mixture was stirred vigorously and refluxed for 4 h. After concentration, the crude product was purified by FC (hexane/EtOAc, 2/1) to afford 0.39 g (48%) of 4 as an oil (α/β=42/58). ¹H NMR (CDCl₃, 400 MHz) a 8.00-7.71 (m, 4H), 7.39-7.34 (m, 2H), 7.29-7.34 (m, 2H), 7.29-7.17 (m, 6H), 5.62-5.58 (m, 1H), 5.34 (t, 1H, α-isomer 1′-H, J=6.8 Hz), 5.23 (dd, 1H, β-isomer 1-H, J=11.2, 5.0 Hz), 4.70-4.52 (m, 3H), 3.87-3.82 (m, 2H), 2.97-2.84 (m, 3H), 2.52 (dd, 1H, B-isomer 2′-Hα, J=13.8, 5.0 Hz), 2.44-2.40 (m, 6H), 2.34-2.20 (m, 1H). ¹³C NMR (CDCl₃, 125 MHz) δ 166.4, 166.1, 166.0, 144.1, 144.0, 143.8, 140.4, 138.8, 138.2, 137.7, 129.8, 129.7, 129.6, 129.2, 129.1, 129.0, 128.9, 127.1, 127.0, 126.8, 126.2, 126.0, 125.9, 81.9, 80.0, 76.4, 64.8, 64.6, 63.6, 63.5, 40.3, 38.8, 21.7, 21.6. MALDI-FTMS: calcd for M+Na⁺497.1934, found 497.1932.

1,4-anhydro-2-deoxy-1-C-[4-[2-1 (methylsulfonyl)oxy]ethyl]phenyl]-D-erythro-pentitol 3,5-bis (4-methylbenzoate) (5). Compound 4 (0.387 g, 0.82 mmol) was dissolved in CH₂Cl₂ (10 ml). Methanesulfonyl chloride (0.126 ml, 1.63 mmol) and then NEt₃ (0.262 ml, 1.88 mmol) were added at 0° C. under nitrogen. The mixture was stirred overnight while the temperature was allowed to rise to room temperature. The CH₂Cl₂ layer was washed with water and brine, and then dried over Na₂SO₄. After evaporation of solvent, 0.435 g (96%) of the product 5 was obtained as a yellow oil and used in the next step without further purification. ¹H NMR (CDCl₃, 500 MHz) δ 7.97-7.72 (m, 4H), 7.40-7.18 (m, 8H), 5.61-5.58 (m, 1H), 5.34 (t, 1H, α-isomer 1′-H, J=7.0 Hz), 5.24 (dd, 1H, β-isomer 1′-H, J=11.0, 5.2 Hz), 4.70-4.52 (m, 3H), 4.42-4.37 (m, 2H), 3.08-3.97 (m, 2H), 2.84 (s, 3H), 2.23 (dd, 1H, β-isomer 2′-Hα, J=14.0, 5.2 Hz), 2.44-2.40 (m, 6H), 2.31-2.19 (m, 1H). ¹³C NMR (CDCl₃, 125 MHz) δ 166.3, 166.0, 144.1, 143.9, 143.8, 141.2, 140.0, 135.9, 135.4, 131.5, 129.6, 129.5, 129.1, 129.0, 128.9, 127.0, 126.9, 126.7, 126.2, 126.0, 82.9, 82.0, 80.4, 80.0, 77.1, 76.3, 70.0, 64.6, 64.5, 53.4, 41.6, 40.3, 37.2, 35.2, 31.4, 21.7, 21.6, 21.5. MALDI-FTMS: calcd for M+Na+575.1710, found 575.1711.

1,4-anhydro-2-deoxy-1-C-[4-(2-azidoethyl) phenyl]-D-erythro-pentitol 3,5-bis (4-methylbenzoate) (6). Compound 5 (0.78 g, 1.43 mmol) was dissolved in anhydrous DMF (15 ml) under nitrogen and then NaN₃ (0.186 g, 2.86 mmol) was added. The mixture was stirred at 40° C. and followed by tlc which showed completion in 4 h. After dilution with EtOAc, aqueous workup and solvent evaporation, the residue was purified by FC (hexane/EtOAc, 3:1). The desired β-isomer of 6 (0.30 g, 42%) eluted first and was obtained as a syrup. ¹H NMR (CDCl₃, 500 MHz) δ 7.98 (d, 2H, J=8.0 Hz), 7.94 (d, 2H, J=8.0 Hz), 7.35 (d, 2H, J=8.0 Hz), 7.27 (d, 2H, J=8.0 Hz), 7.22 (d, 2H, J=7.7 Hz), 7.18 (d, 2H, J=7.7 Hz), 5.61 (d, 1H, J=5.5 Hz), 5.24 (dd, 1H, J=10.6, 4.8 Hz), 4.65-4.64 (m, 2H), 4.54-4.52 (m, 1H), 3.49 (t, 2H, J=7.4 Hz), 2.88 (t, 2H, J-7.4 Hz), 2.52 (dd, 1H, J=14.0, 5.2 Hz), 2.44 (s, 3H), 2.41 (s, 3H), 2.27-2.20 (m, 1H). ¹³CNMR (CDCl₃, 100 MHz) δ 166.3, 166.1 144.1, 143.8, 139.1, 137.6, 129.7, 129.6, 129.2, 129.1, 128.8, 127.0, 126.9, 126.2, 82.9, 80.6, 77.2, 64.7, 52.3, 41.6, 34.9, 21.7, 21.6. MALDI-FTMS: calcd for M+Na⁺ 522.1999, found 522.1997.

1,4-anhydro-2-deoxy-1-C-[4-(2-aminoethyl) phenyl]-D-erythro-pentitol 3,5-bis (4-methylbenzoate) (7). Compound 6 (0.357 g, 0.71 mmol) was dissolved in THF (10 ml). Ph₃P (0.28 g, 1.06 mmol) and water (0.1 ml) were added. The reaction mixture was stirred at room temperature under nitrogen for 36 h until tlc showed the disappearance of starting material. The mixture was concentrated and the residue purified by FC (hexane/EtOAc, 1/2) to remove Ph₃P, Ph₃PO and by-products, and then (CH₂Cl₂/MeOH, 3/1) to give 0.30 g (83%) of 7 as a yellow syrup. ¹H NMR (CDCl₃, 400 MHz) δ 7.98 (d, 2H, J=8.2 Hz), 7.94 (d, 2H, J=8.2 Hz), 7.33 (d, 2H, J=8.2 Hz), 7.27 (d, 2H, J=8.2 Hz), 7.22 (d, 2H, J=7.9 Hz), 7.16 (d, 2H, J=7.9 Hz), 5.62-5.60 (m, 1H), 5.23 (dd, 1H, J=10.8, 5.0 Hz), 4.65-4.64 (m, 21), 4.53 (brs, 1H), 2.95 (brs, 2H), 2.74 (t, 2H, J=7.0 Hz), 2.51 (dd, 1H, J=13.8, 5.0 Hz), 2.43 (s, 3H), 2.40 (s, 3H), 2.28-2.20 (m, 1H). ¹³C NMR (CDCl₃, 100 MHz) δ 166.3, 166.1, 144.1, 143.8, 139.4, 138.4, 129.6, 129.1, 129.0, 128.9, 127.0, 126.9, 126.1, 125.9, 82.8, 80.7, 77.2, 64.7, 43.3, 41.6, 39.4, 21.7, 21.6. MALDI-FTMS: calcd for M+Na⁺ 496.2094, found 496.2100.

N-[2-[4-[2-deoxy-3,5-bis-O-(4-methylbenzoyl)-D-erythro-pentofuranosyl]phenyl]ethyl]-N′-[4-[(1E)-2-phenylethenyl]phenyl]-pentanediamide (8). Into a mixture of 7 (163 mg, 0.345 mmol) and 12 (117 mg, 0.379 mmol) in DMF (3.5 ml) was added EDC-HCl (88 mg, 0.448 mmol) at room temperature. The mixture was stirred under nitrogen for 4 h. After concentration, the residue was purified by FC (EtOAC) to afford 172 mg (65%) of 8 as a syrup. ¹H NMR (CDCl₃, 400 MHz) δ 8.50 (s, 1H), 7.97 (d, 2H, J=8.2 Hz), 7.94 (d, 2H, J=8.2 Hz), 7.56 (d, 2H, J=8.5 Hz), 7.49-7.42 (m, 4H), 7.36-7.12 (m, 1H), 7.02 (dd, 2H, J=20.0, 16.4 Hz), 5.91 (t, 1H, J=6.2 Hz), 5.59 (d, 1H, J=6.8 Hz), 5.20 (dd, 1H, J=10.9, 5.0 Hz), 4.67-4.59 (m, 2H), 4.52-4.52 (m, 1H), 3.52-3.51 (m, 2H), 2.80 (t, 2H, J=7.0 Hz), 2.50 (dd, 1H, J=14.1, 5.9 Hz), 2.42 (s, 3H), 2.40 (s, 3H), 2.35-2.31 (m, 2H), 2.62-2.23 (m, 2H), 1.98-1.92 (m, 2H). ¹³C NMR (CDCl₃, 100 MHz) 172.8, 171.1, 166.4, 166.1, 144.2, 143.9, 138.7, 138.5, 137.6, 137.3, 133.0, 129.7, 129.2, 128.9, 128.6, 127.6, 127.4, 127.0, 126.9, 126.4, 126.3, 119.8, 82.9, 80.6, 77.1, 64.7, 41.4, 40.4, 36.1, 35.2, 35.0, 21.8, 21.6. MALDI-FTMS: calcd for M+Na⁺ 787.3354, found 787.3334.

N-[2-[4-[2-deoxy-D-erythro-pentofuranosyl] phenyl]ethyl]-N′-[4-[(1E)-2-phenylethenyl]phenyl]-pentanediamide (9). Compound 8 (172 mg, 0.225 mmol) was dissolved in MeOH/CH₂Cl₂ (3 ml/2 ml) at room temperature under nitrogen. A solution of 25% MeONa in MeOH (0.154 ml, 0.675 mmol) was added with stirring. After 30 min, a suspension developed and tlc indicated the disappearance of starting material. After stirring for an additional 1.5 h, solid NH₄Cl was added to quench the reaction followed by water (1 ml). The solid was collected by filtration, washed with water and dried under vacuum in a desiccator to afford 94 mg (76%) of 9 as a white powder. ¹H NMR CDMSO-d₆, 500 MHz) δ 9.92 (brs, 1H), 7.86 (brs, 1H), 7.61 (d, 2H, J=8.4 Hz), 7.56 (d, 2H, J=7.4 Hz), 7.52 (d, 2H, J=8.4 Hz), 7.35 (t, 2H, J=7.7 Hz), 7.26-7.22 (m, 3H), 7.19-7.11 (m, 4H), 4.96-4.94 (m, 2H), 4.69 (brs, 1H), 4.16 (brs, 1H), 4.76-4.75 (m, 1H), 3.49-3.38 (m, 2H), 3.26-3.23 (m, 2H), 2.68 (t, 2H, J=7.4 Hz), 2.31 (t, 2H, J=7.4 Hz), 2.11 (t, 2H, J=7.0 Hz), 2.03 (dd, 1H,J=12.4, 5.5 Hz), 1.83-1.72 (m, 3H). ¹³C NMR (DMSO-d₆, 125 MHz) δ 171.5, 170.8, 140.3, 138.8, 138.4, 137.2, 131.7, 128.6, 128.3, 128.0, 127.3, 126.8, 126.2, 126.1, 119.1, 87.7, 79.0, 72.4, 62.5, 43.4, 35.7, 34.9, 34.6, 21.1. MALDI-FTMS: calcd for M+Na⁺ 551.2516, found 551.2524.

5-oxo-5-[[4-[(1E)-2-phenylethenyl]phenyl]amino]-pentanoic acid (12). A solution of trans-4-aminostilbene 11 (0.53 g, 2.7 mmol) (TCI Chem. Co.) in CH₂Cl₂ (10 ml) was stirred at room temperature and triethylamine (1.13 ml, 7.1 mmol) was added followed by glutaric anhydride (464 mg, 4.05 mmol) and 4-dimethylaminopyridine (DMAP) (2 mg). The solution was stirred at room temperature for 18 h, poured into water/EtOAc, shaken, filtered and the solid washed with water, EtOAc, hexane, and then dried that afforded 12 as a white solid (340 mg, 41%). ¹H NMR (DMSO-d₆, 500 MHz): δ 9.96 (1H, s), 7.60 (2H, d, J=8.8 Hz), 7.56 (2H, d, J=7.4 Hz), 7.52 (2H, d, J=8.8 Hz), 7.35 (2H, t, J=7.4 Hz), 7.23 (1H, t, J=7.4 Hz), 7.18 (1H, d,J=16.5 Hz), 7.13 (1H, d,J=16.5 Hz), 2.36 (2H, t,J=7.3 Hz), 2.27 (2H, t, J=7.3 Hz), 1.81 (2H, quin, J=7.3 Hz). ³C NMR (DMSO-d₆, 125 MHz): δ 174.12, 170.69, 138.80, 137.22, 131.78, 128.65, 128.03, 127.30, 126.88, 126.84, 126.23, 119.10, 35.39, 32.96, 20.39. MALDI-FTMS: calcd for C₁₉H₁₉NO₃ 332.1263 (M+Na⁺), found: 332.1256.

4-[(methoxymethoxy) methyl]bromobenzene (14). To a solution of 4-bromobenzyl alcohol 13 (4.0 g, 21.4 mmol) in dimethoxymethane (40 ml) was added LiBr (0.37 g, 4.28 mmol) andp-TsOH—H₂O (0.41 g, 2.14 mmol). The white suspension was stirred at room temperature for 2 h, then quenched by addition of brine, followed by extraction of the mixture with ether. After evaporation, the residue was purified by FC (hexane/EtOAc, 4/1) to give 14 (4.0 g, 81%) as a colorless oil. ¹H NMR (CDCl₃, 400 MHz) δ 7.47 (d, 2H, J=8.0 Hz), 7.23 (d, 2H, J=8.0 Hz), 4.69 (s, 2H), 4.54 (s, 2H), 3.40 (s, 3H). ¹³C NMR (CDCl₃, 100 MHz) δ 136.8, 131.4, 129.4, 121.4, 95.6, 68.3, 55.3.

1,4-anhydro-2-deoxy-1-C-[4-[(methoxymethoxy) methyl]phenyl]-3,5-O-[1,1,3, 3-tetrakis (1-methylethyl)-1,3-disiloxanediyl]-D-erythro-pentitol (15). To a solution of compound 14 (0.23 g, 1.0 mmol) in dry THF (2.5 ml) at −78° C. under N₂ was added t-BuLi (1.7 M in pentane, 1.17 ml, 2.0 mmol). The mixture was stirred for 30 min and then transferred to a solution of 19 (0.224 g, 0.60 mmol) in dry THF (2.5 ml) at −78° C. After 1 h, the reaction was quenched with sat. aq. NH₄Cl and the mixture extracted with ether. The organic layer was washed with water and brine, dried over Na₂SO₄ and the solvent evaporated to give a crude oil. To a solution of the oil at −78° C. in CH₂Cl₂ (5 ml) under N₂ was added Et₃SiH (0.288 ml, 1.8 mmol) and BF₃-Et₂O (0.227 ml, 1.8 mmol). The mixture was stirred at −78° C. for 6 h and then quenched by addition of sat. NaHCO₃ at −78° C. The mixture was extracted with ether and the ether layer was washed with water, brine and dried over Na₂SO₄. After evaporation, the crude oil was purified by FC (hexane/EtOAc, 8/1) to give product 15 (0.13 g, 42%) as a colorless oil. ¹H NMR (CDCl₃, 500 MHz) δ 7.33 (s, 4H), 5.10 (t, 1H, J=7.3 Hz), 4.70 (s, 2H), 4.58 (s, 2), 4.56-4.52 (m, 1H), 4.15 (d, 1H, J=8.4 Hz), 3.94-3.87 (m, 2H), 3.41 (s, 3H), 2.40-2.35 (m, 1H), 2.09-2.03 (m, 1H), 1.14-0.95 (m, 28H). ¹³C NMR (CDCl₃, 125 MHz) δ 141.6, 137.0, 127.9, 125.8, 95.5, 86.3, 78.8, 73.2, 68.8, 63.6, 55.2, 43.1, 17.5, 17.4, 17.3, 17.2, 17.0, 16.9, 13.4, 13.3, 12.9, 12.5. MALDI-FTMS: calcd for M+Na⁺ 533.2725, found 533.2725.

1,4-anhydro-2-deoxy-1-C-[4-(hydroxymethyl) phenyl]-3,5-O-[1,1,3,3-tetrakis (1-methylethyl)-1,3-disiloxanediyl]-1)-erythro-pentitol (16). To a solution of compound 15 (0.121 g, 0.237 mmol) in CH₂Cl₂ (5 ml) at −30° C. under N₂ was added TMSBr (0.125 ml, 0.949 mmol). After stirring at −30° C. for 1 h, the reaction was quenched by addition of sat. NaHCO₃ and the mixture extracted with ether. After evaporation, the crude oil was purified by PTLC (hexanes/EtOAc, 1/1) to give 16 (43 mg, 390%) as a colorless oil. ¹H NMR (CDCl₃, 400 M z) δ 7.34 (s, 4H), 5.09 (t, 1H, J=7.0 Hz), 4.68 (s, 2H), 4.554.51 (m, 1H), 4.13 (dd, 1H, J=10.3, 2.1 Hz), 4.93-3.86 (m, 2H), 2.37-2.34 (m, 1H), 2.06 (dt, 1H, J=12.9, 7.6 Hz), 1.12-0.94 (m, 28H). ¹³C NMR (CDCl₃, 125 MHz) δ 141.6, 127.1, 126.1, 86.4, 78.8, 73.2, 65.2, 63.6, 43.1, 17.6, 17.4, 17.3, 17.2, 17.1, 17.0, 13.5, 13.4, 13.0, 12.5. MALDI-FTMS: calcd for M+Na⁺ 489.2463, found 489.2457.

1,4-anhydro-2-deoxy-1-C-[4-[[2-[2-[[4-[(1E)-2-phenylethenyl]phenyl]methoxy]ethoxy]ethoxy]methyl]] phenyl]-3,5-O-[1,1,3,3-tetrakis (1-methylethyl)-1,3-disiloxanediyl]-D-erythro-pentitol (17). Triflic anhydride (0.0176 ml, 0.105 mmol) was added to dry CH₂Cl₂ (0.5 ml) at −70° C. under N₂ followed by a solution of 16 (46.7 mg, 0.10 mmol) and 2,4,6-collidine (0.0139 ml, 0.105 mmol) in CH₂Cl₂ (1 ml). After 30 min, a solution of 23 (29.8 mg, 0.10 mmol) and 2,4,6-collidine (0.0264 ml, 0.20 mmol) in CH₂Cl₂ (1 ml) was added with stirring. After 30 min, the mixture was allowed to warm to room temperature for an additional 3 h. The reaction mixture was concentrated and purified by PTLC (hexane/EtOAc, 2/1) to give 17 (17.9 mg, 24%) as a colorless oil. ¹H NMR (CDCl₃, 400 MHz) δ 7.53-7.48 (m, 4H), 7.38-7.24 (m, 9H), 7.10 (s, 2H), 5.08 (t, 2H, J=8.0 Hz), 4.58 (s, 2H), 4.56 (s, 2H), 4.53-4.50 (m, 1H), 4.13 (d, 2H, J=8.0 Hz), 3.91-3.85 (m, 2H), 3.71-3.62 (m, 8H), 2.38-2.32 (m, 1H), 2.09-2.02 (m, 1H), 1.11-1.01 (m, 28H). ¹³C NMR (CDCl₃, 100 MHz) δ 141.4, 137.7, 137.5, 137.3, 128.6, 128.5, 128.3, 128.1, 127.8, 127.6, 126.5, 125.9, 86.4, 78.9, 73.3, 73.0, 70.7, 69.4, 69.3, 63.7, 43.1, 17.6, 17.4, 17.4, 17.2, 17.1, 17.0, 13.5, 13.4, 13.0, 12.5. MALDI-FTMS: calcd for M+Na+769.3926, 769.3914.

1,4-anhydro-2-deoxy-1-C-[4-[[2-[2-[[4-[(1E)-2-phenylethenyl] phenyl]methoxy]ethoxy]ethoxy]methyl]]phenyl]-D-erythro-pentitol (18). To a solution of 17 (17.9 mg, 0.024 mmol) in THF (0.3 ml) at 0° C. under N₂ was added TBAF (1.0 M in THF, 0.072 ml, 0.05 mmol). The mixture was stirred for 2 h while the reaction temperature was allowed to warm to room temperature. After concentration, the crude oil was purified by PTLC (EtOAc/MeOH, 40/1) to give 18 (11.8 mg, 98%) as a white syrup. ¹H NMR (CDCl₃, 400 MHz) δ 7.53-7.48 (m, 4H), 7.38-7.24 (m, 9), 7.10 (s, 2H), 5.16 (dd, 1H, J=10.0, 5.0 Hz), 4.58(s, 2H), 4.57 (s, 2H), 4.42-4.40 (m, 1H), 4.01-3.99 (m, 1H), 3.81 (dd, 1H, J=12.0, 4.0 Hz), 3.73-3.63 (m, 9H), 2.23 (ddd, 1H, J=13.2, 5.5, 1.8 Hz), 2.05-1.98 (m, 1H), 1.93 (brs, 2H). ¹³C NMR (CDCl₃, 100 MHz) δ 140.9, 138.3, 138.1, 137.7, 137.1, 129.1, 129.0, 128.8, 128.6, 128.4, 128.0, 126.9, 126.5, 87.6, 80.3, 74.2, 73.4, 73.3, 71.1, 69.9, 63.8, 44.5. MALDI-FTMS: calcd for M+Na⁺ 527.2404, found 527.2402.

4-chloromethyl-trains-stilbene (21). To a solution of 4-hydroxymethyl-trans-stilbene 20 (0.557 g, 2.65 mmol) and triethylamine (0.85 ml, 6.1 mmol) in CH₂Cl₂ (30 ml) at 0° C. was added MsCl (0.41 ml, 5.3 mmol) dropwise with stirring. The reaction mixture was stirred at room temperature overnight. After work-up, 21 (0.602 g, 99%) was obtained as a white solid. ¹H NMR (CDCl₃, 400 MHz) δ 7.54-7.50 (m, 4H), 7.40-7.35 (m, 4H), 7.30-7.28 (m, 1H), 7.12 (d, 2H, J=2.3 Hz), 4.61 (s, 2H).

11-[4-[(1E)-2-phenylethenyl] phenyl]-2, 4, 7, 10-tetraoxaundecane (22). The alcohol 26 (0.40 g, 2.65 mmol) was treated with 60% NaH (0.19 g, 4.75 mmol) in dry THF (10 ml) at room temperature for 10 min. To this mixture, a solution of 21 (0.602 g, 2.65 mmol) in THF (10 ml) and cat. NaI was added. The mixture was stirred at 60° C. overnight. The reaction was quenched by addition of water and the mixture extracted with ether. After evaporation, the crude oil was purified by FC (hexane/EtOAc, 2/1) to give 22 (0.7 g, 77%) as a yellow oil. ¹H NMR (CDCl₃, 500 MHz) 8753-7.49 (m, 4H), 7.38-7.34 (m, 4H), 7.28-7.25 (m, 1H), 7.11 (s, 2H), 4.68 (s, 2H), 4.58 (s, 2H), 3.74-3.66 (m, 8H), 3.38 (s, 3H). 3 NMR (CDCl₃, 100 MHz) δ 137.6, 137.2, 136.6, 128.6, 128.5, 128.3, 128.1, 127.6, 126.4, 96.5, 72.9, 70.6, 70.5, 69.4, 66.7, 55.1. MALDI-FTMS: calcd for M+Na+365.1723, found 365.1716.

2-[2-[[4-[(1E)-2-phenylethenyl]phenyl]methoxy]ethoxy]-ethanol (23). A solution of 22 (0.7 g, 2.05 mmol) in MeOH (10 ml) was treated with a catalytic amount of conc. HCl at 65° C. The reaction was followed by TLC until starting material disappeared (8 h). The mixture was concentrated and purified by FC (hexane/EtOAc, 1/1) to give 23 (0.524 g, 86%) as a pale white solid. ¹H NMR (CDCl₃, 400 MHz) δ 7.54-7.50 (m, 4H), 7.39-7.34 (m, 4H), 7.29-7.26 (m, 1H), 7.12 (s, 2H), 4.57 (s, 2H), 3.75-3.73 (m, 2H), 3.71-3.68 (m, 2H), 3.66-3.64 (m, 2H), 3.62-3.60 (m, 2H), 3.02 (brs, 1H). ¹³C NMR (CDCl₃, 100 MHz) δ 137.2, 137.0, 136.6, 128.5, 128.0, 127.9, 127.4, 126.3, 72.8, 72.3, 70.2, 69.2, 61.5. MALDI-FTMS: calcd for M+Na⁺ 321.1461, found 321.1454.

Di (ethyleneglycol) benzyl methoxymethyl ether (25). To a solution of di(ethyleneglycol) benzyl ether 24 (2.0 g, 10.0 mmol) in dimethoxymethane (20 ml) was added LiBr (0.17 g, 2.0 mmol) andp-TsOH—H₂O (0.19 g, 1.0 mmol). The white suspension was stirred at room temperature for 3 h, then the reaction was quenched by addition of brine and the mixture extracted with ether. After evaporation, the residue was purified by FC (Hexanes/AcOEt 3/1) to give 25 (1.92 g, 80%) as a colorless oil. ¹H NMR (CDCl₃, 400 MHz) δ 7.35-7.26 (m, 5H), 4.67 (s, 2H), 4.58 (s, 2H), 3.72-3.64 (m, 8H), 3.37 (s, 3H). ¹³C NMR (CDCl₃, 125 MHz) δ 138.2, 128.3, 127.6, 127.5, 96.5, 73.2, 70.6, 70.5, 69.4, 66.8, 55.1.

Di (ethyleneglycol) methoxymethyl ether (26). The benzylether 25 (1.92 g, 8.0 mmol) was dissolved in chloroform (10 ml) with 10% Pd/C (0.85 g, 0.1 mmol) and stirred under a hydrogen atmosphere provided by a balloon. The reaction was followed by TLC and was complete in 1 h. The mixture was filtered and the filtrate concentrated to give 26 (1.12 g, 93%) as a colorless oil. ¹H NMR (CDCl₃, 400 MHz) δ 4.68 (s, 2H), 3.76-3.62 (m, 8H), 3.38 (s, 3H). ¹³C NMR (CDCl₃, 125 MHz) δ 96.5, 72.4, 70.4, 66.9, 61.7, 55.2.

X-ray crystallography. Crystals of mAb 19G2 were soaked overnight with a 0.25 mM solution of either 9 or 18 (DMF stock solutions) in mother liquor from the crystal growth (12% polyethylene glycol, 0.1 M sodium acetate pH 4.75, 0.3 M magnesium chloride) containing 5% DMF. Formation of the complex in the crystal was assayed by the appearance of blue fluorescence from soaked crystals when illuminated by a hand-held UV lamp at 312 nm (Spectronics Corp.; Westbury, N.Y.). The crystals were soaked in a cryobuffer consisting of 20% glycerol, 0.25 mM 9 or 18, mother liquor, and 5% DMF and flash frozen in liquid nitrogen. X-ray diffraction was collected in-house with an FRD X-ray generator and a RAXISIVF⁺⁺ detector. Data was processed and scaled using HKL software package. The previously determined structure of the antibody (PDB ID CODE 1FL3) was used as a starting model for refinement. Multiple rounds of rigid body refinement, B-factor refinement, Powell minimization, simulated annealing in CNS and manual rebuilding in O were performed.

What is claimed is:

1. An anti-stilbene antibody.

2. The antibody of claim 1 that is a monoclonal antibody.

3. The antibody of claim 1 designated 19G2, 20F2, 21C6, 22B9, 25F8, 25E2, 23E4, 23G3, 23D3, 23C2, 25C10, 24B6, 21E2, 16H10 and 9E11.

4. A hybridoma that produces the antibody of claim 3.

5. A method of detecting stilbene comprising the step of exposing stilbene to an anti-stilbene antibody and detecting an anti-stilbene/antibody-stilbene conjugate.

6. The method of claim 5 wherein detecting is accomplished by exciting the conjugate with light of a wavelength of from about 300 nm to about 350 nm and detecting emitted light having a wavelength of from about 380 nm to about 410 nm.

7. The method of claim 5 wherein the stilbene is attached to a target moiety.

8. The method of claim 7 wherein the target moiety is a polypeptide or polynucleotide.

9. The method of claim 7 wherein the target moiety is located in vivo.

10. A nucleoside that contains stilbene.
